(19) United States
(12) Patent Application Publication
(10) Pub. No.: US 2025/0154161 A1
(43) Pub. Date: May 15, 2025

(54) CRYSTAL FORMS OF THIENOIMIDAZOLE COMPOUND AND PREPARATION METHOD THEREOF

(71) Applicant: HANGZHOU SCIWIND BIOSCIENCES CO., LTD., Hangzhou Zhejiang (CN)

(72) Inventors: Lu GAN, Shanghai (CN); Tao YU, Shanghai (CN); Chengde WU, Shanghai (CN); Ting YAO, Shanghai (CN)

(73) Assignee: HANGZHOU SCIWIND BIOSCIENCES CO., LTD., Hangzhou Zhejiang (CN)

(21) Appl. No.: 18/715,503

(22) PCT Filed: Dec. 2, 2022
(86) PCT No.: PCT/CN2022/136097

§ 371 Date: May 31, 2024

(30) Foreign Application Priority Data
Dec. 3, 2021 (CN) 202111474324.5

(51) Int. Cl. C07D 495/04

(52) U.S. Cl. CPC C07D 495/04

(57) ABSTRACT

The application relates to crystal forms of thienoimidazole compound and preparation method thereof, and use of the crystal forms in the preparation of a medicament for treating related diseases. Formula (II) compound, Formula (III) compound, Formula (IV) compound, Formula (V) compound, Formula (VI) compound, Formula (VII) compound and Formula (VIII) compound.

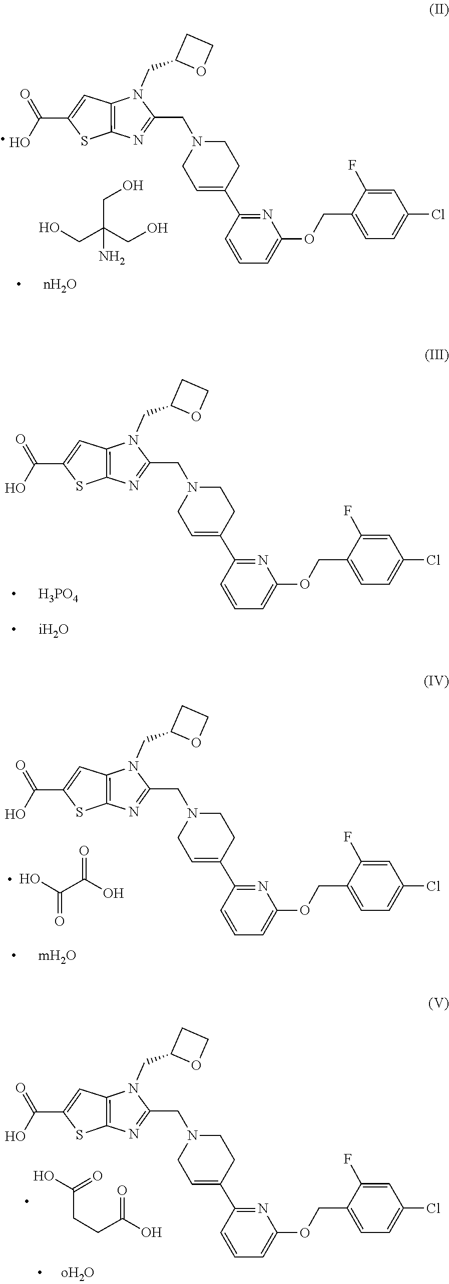

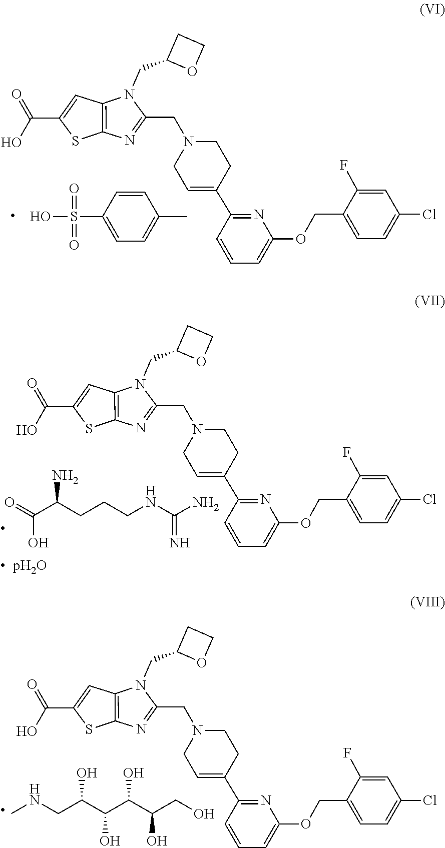

### TECHNICAL FIELD

The application relates to crystal forms of thienoimidazole compound and preparation method thereof, and use of the crystal forms in the preparation of a medicament for treating related diseases.

### BACKGROUND ART

Diabetes is a common metabolic disease characterized by hyperglycemia. Several major types of diabetes are caused by complex interactions between genetic and environmental factors. The factors that lead to hyperglycemia include the decrease in insulin secretion, the decrease in glucose utilization and the increase in glucose output, and the dominance of these factors varies with the etiology of diabetes. Metabolic abnormalities related to diabetes cause secondary pathophysiological changes in multiple systems of the body. Long-term abnormal blood sugar level can lead to serious complications, including cardiovascular disease, chronic renal failure, retinal injury, nerve injury, microvascular injury, obesity and the like. The classification of diabetes is based on the different pathological processes that lead to hyperglycemia. Diabetes can be divided into two main types: type 1 diabetes and type 2 diabetes. In the process of disease development, Type 1 and Type 2 diabetes have abnormal glucose homeostasis before onset. Type 1 diabetes is caused by complete or almost complete insulin deficiency. Type 2 diabetes is a group of heterogeneous diseases, characterized by varying degrees of insulin resistance, decreased insulin secretion, and increased glucose production. For the treatment of diabetes, in the early stage, diet control and exercise therapy are the first choice for blood sugar control. When these methods are difficult to achieve the control of blood sugar, insulin or oral hypoglycemic drugs should be used for patients. At present, drugs used for the treatment of diabetes include insulin, insulin secretagogues, metformin, insulin sensitizers, a-glucosidase inhibitors, dipeptidyl peptidase-IV inhibitors (-gliptin), sodium-glucose cotransporters 2 (SGLT2) inhibitors, glucagon like peptide-1 (GLP-1) receptor agonists and the like. These drugs have good therapeutic effects, but there are still safety problems in long-term treatment, for example, biguanides are easy to cause lactic acidosis; sulfonylureas can cause hypoglycemia symptoms; insulin sensitizers can cause edema, heart failure and weight gain; a-glucosidase inhibitors can cause abdominal pain, abdominal distension, diarrhea and other symptoms; sodium-glucose cotransporters 2 (SGLT2) inhibitors increase the risk of urinary and reproductive system infections. Therefore, there is an urgent need to develop a safer and more effective new hypoglycemic drug to meet the treatment needs of diabetes.

Glucagon-like peptide-1 receptor (GLP-1R) is one of the most important therapeutic targets for type 2 diabetes. GLP-1R is a member of G protein coupled receptor B cluster subfamily, which is widely expressed in stomach, small intestine, heart, kidney, lung, brain and other tissues. In islet cells, GLP-1R mainly promotes the release of insulin, increases the regeneration of islet B cells, inhibits the apoptosis of B cells, and reduces the release of glucagon. In gastrointestinal tract and other tissues, GLP-1R can inhibit gastrointestinal peristalsis and gastric juice secretion by combining with its agonists, delay gastric emptying and increase satiety. In neural tissue, small molecule GLP-1R agonists can penetrate into the brain to activate the subset of neurons expressing GLP-1R, protect neuronal from apoptosis and enhance learning and memory ability. Additionally, GLP-1R can also control food intake to reduce weight. GLP-1 receptor agonists or endogenous GLP-1 activity enhancers are approved for the treatment of type 2 diabetes. These drugs do not cause hypoglycemia, because the insulin secretion stimulated by secretin is glucose dependent. Exenatide is a kind of synthetic peptide, which was originally found in the saliva of Heloderma and is a GLP-1 analog. Compared with natural GLP-1, exenatide has different amino acid sequence, which makes it resistant to the enzyme degrading GLP-1 [dipeptidyl peptidase IV (DPP-IV)]. Therefore, exenatide has an extended GLP-1 like activity, and can bind to GLP-1 receptors in the pancreatic islets, gastrointestinal tract and brain. Liraglutide, another GLP-1 receptor agonist, is almost the same as the natural GLP-1 except that it replaces one of the amino acids and adds one fatty acyl group; and this fatty acyl group can promote its binding with albumin and plasma protein and prolong the half-life. GLP-1 receptor agonist can increase the insulin secretion stimulated by glucose, inhibit glucagon, and delay gastric emptying. These drugs do not increase weight. In fact, most patients will lose weight and appetite to some extent.

DPP-IV inhibitors inhibit the degradation of natural GLP-1, thereby enhancing the effect of secretin. DPP-IV, which is fully expressed on the cell surface of endothelial cells and some lymphocytes, can degrade a variety of polypeptides (not only GLP-1). DPP-IV inhibitors promote insulin secretion without lowering blood sugar, do not gain weight, and have more advantages in reducing postprandial blood sugar. Patients using GLP-1 receptor agonists had higher level of GLP-1 activity than patients using DPP-IV inhibitors.

The development of a small molecule GLP-1 receptor agonist with oral activity can effectively avoid long-term self-injection of patients, with good compliance. Small molecule GLP-1 receptor agonists can control blood sugar through multiple pathways including glucose metabolism and excretion, which is expected to develop safer and more effective new hypoglycemic drugs to meet the treatment needs of diabetes.

### SUMMARY OF THE APPLICATION

The present application provides a pharmaceutically acceptable salt of the formula (O) compound,

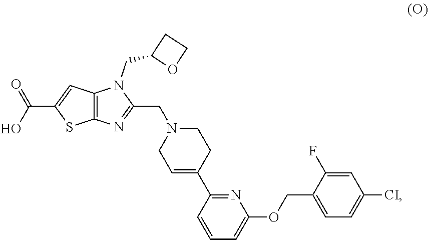

wherein the pharmaceutically acceptable salt is selected from the group consisting of: trometamol salt, phosphate, citrate, oxalate, maleate, L-tartrate, p-toluenesulfonate N, sodium salt, potassium salt, L-arginine salt, hydroxycholine salt, meglumine salt; preferably trometamol salt, phosphate, oxalate, maleate, p-toluenesulfonate N, L-arginine salt, meglumine salt; more preferably trometamol salt, meglumine salt.

In some embodiments of the application, the chemical molar ratio of the compound to acid or base molecule is 1:2-2:1, for example, the chemical molar ratio may be 2:3, 3:4, 4:5, 1:1, 5:4, 4:3, or 3:2. In some embodiments, preferably the chemical molar ratio may be 1:2-1:1. In some embodiments, preferably the chemical molar ratio may be 1:1-2:1. In some embodiments, preferably the chemical molar ratio may be 1:2, 1:1, or 2:1.

The present application provides a method for preparing the above pharmaceutically acceptable salt, and the method comprises the step of salt forming reaction of the formula (I) compound and acid or base.

In some embodiments of the application, the solvent used in the salt forming reaction is at least one selected from the group consisting of: butanone, methanol, ethanol, tetrahydrofuran, ethyl acetate, isopropanol, acetonitrile, and methyl tert-butyl ether.

The present application provides a pharmaceutical composition comprising the above pharmaceutically acceptable salt and optionally a pharmaceutically acceptable carrier or excipient.

In some embodiments of the application, provided is use of the above pharmaceutically acceptable salt in the preparation of a medicament for preventing and/or treating a metabolic disease, wherein preferably the metabolic disease is diabetes, obesity, or nonalcoholic fatty liver disease.

The present application provides formula (II) compound

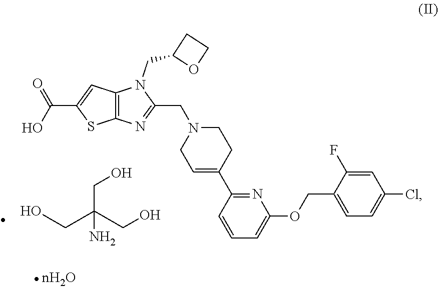

wherein n is selected from 0-12, for example, it may be 0, ½, ⅔, 1, 2, 2.7, 3, 4, 5, 6, 7, or 8. In some embodiments of the application, n is selected from 0-8. In some embodiments of the application, n is selected from 0-5. In some embodiments of the application, n is selected from 0-3.

In some embodiments of the application, n is selected from 0, ½, ⅔, 1, 2, 2.7 and 3.

In some embodiments of the application, the formula (II) compound has the following structures:

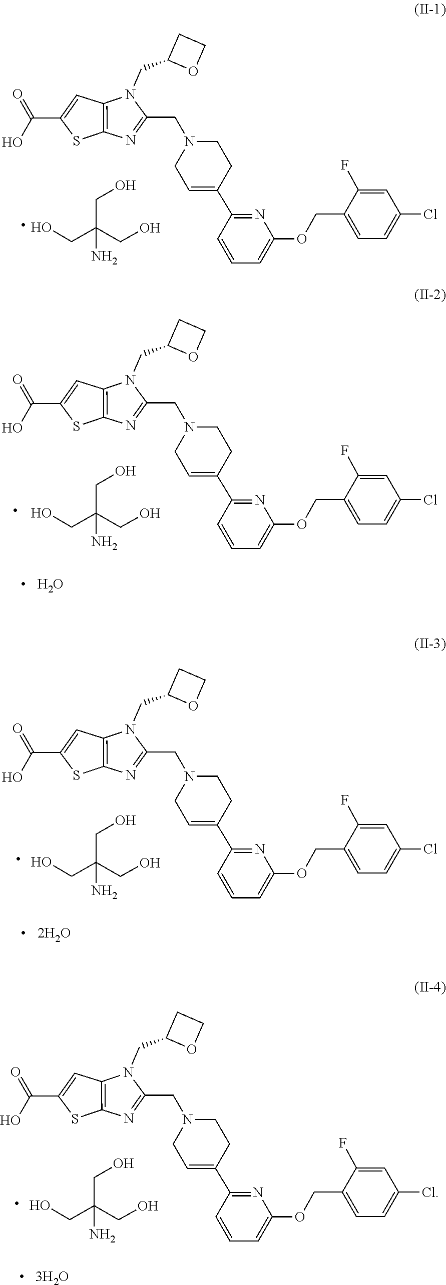

The present application provides crystal Form A of formula (II-1) compound, characterized in that the X-ray powder diffraction pattern of the crystal Form A has characteristic diffraction peaks at the following 2θ angles: 11.1426±0.2000°, 14.4804±0.2000°, and 21.4921±0.2000°.

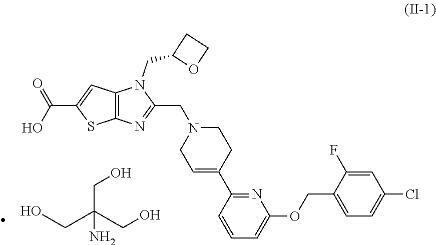 [SMILES: NC(CO)(CO)CO ; O=C(O)c1cc2c(nc(CN3CC=C(c4cccc(OCc5ccc(Cl)cc5F)n4)CC3)n2C[C@@H]2CCO2)s1]

In some embodiments of the application, the X-ray powder diffraction pattern of the above crystal Form A has characteristic diffraction peaks at the following 2θ angles: 11.1426±0.2000°, 14.4804±0.2000°, 17.7642±0.2000°, 19.6235±0.2000°, and 21.4921±0.2000°.

In some embodiments of the application, the X-ray powder diffraction pattern of the above crystal Form A has characteristic diffraction peaks at the following 2θ angles: 11.1426±0.2000°, 14.4804±0.2000°, 17.0921±0.2000°, 17.7642±0.2000°, 19.6235±0.2000°, 20.4359±0.2000°, 21.4921±0.2000°, and 22.8874±0.2000°.

In some embodiments of the application, the X-ray powder diffraction pattern of the above crystal Form A has characteristic diffraction peaks at the following 2θ angles: 5.6759°, 10.2572°, 11.1426°, 14.4804°, 16.2701°, 17.0921°, 17.7642°, 19.6235°, 20.4359°, 20.9310°, 21.4921°, 22.8874°, 25.1971°, 26.5995°, 27.9955°, 28.6836°, 29.4212°, and 30.9896°.

In some embodiments of the application, the X-ray powder diffraction pattern of the above crystal Form A is substantively shown in FIG. 1.

In some embodiments of the application, the analysis data of the XRPD pattern of the above crystal Form A is shown in Table 1:

TABLE 1
Analysis data of the XRPD pattern for crystal
Form A of formula (II-1) compound

| | 2θ | Interplanar | Relative |
|---|---|---|---|
| No. | angle (°) | spacing (Å) | intensity (%) |
| 1 | 5.6759 | 15.57 | 28.57 |
| 2 | 10.2572 | 8.62 | 15.90 |
| 3 | 11.1426 | 7.94 | 85.37 |
| 4 | 14.4804 | 6.12 | 100.00 |
| 5 | 16.2701 | 5.45 | 11.57 |
| 6 | 17.0921 | 5.19 | 50.62 |
| 7 | 17.7642 | 4.99 | 61.88 |
| 8 | 19.6235 | 4.52 | 68.27 |
| 9 | 20.4359 | 4.35 | 41.77 |
| 10 | 20.9310 | 4.24 | 39.32 |
| 11 | 21.4921 | 4.13 | 94.06 |
| 12 | 22.8874 | 3.89 | 42.76 |
| 13 | 25.1971 | 3.53 | 21.49 |
| 14 | 26.5995 | 3.35 | 16.19 |
| 15 | 27.9955 | 3.19 | 18.23 |
| 16 | 28.6836 | 3.11 | 13.89 |
| 17 | 29.4212 | 3.04 | 10.29 |
| 18 | 30.9896 | 2.89 | 7.45 |

In some embodiments of the application, the differential scanning calorimetric curve of the above crystal Form A has a peak value of endothermic peak at 76.0±3.0° C. and 130.3±3.0° C., respectively.

In some embodiments of the application, the DSC thermogram of the above crystal Form A is substantively shown in FIG. 2.

In some embodiments of the application, the thermogravimetric analysis curve of the above crystal Form A has a weight loss of 2.43% at 150.0±3.0° C.

In some embodiments of the application, the TGA spectrum of the above crystal Form A is substantively shown in FIG. 3.

The present application provides crystal Form B of formula (II) compound, characterized in that the X-ray powder diffraction pattern of the crystal Form B has characteristic diffraction peaks at the following 2θ angles: 13.3946±0.2000°, 16.0867±0.2000°, and 18.7923±0.2000°.

In some embodiments of the application, the X-ray powder diffraction pattern of the above crystal Form B has characteristic diffraction peaks at the following 2θ angles: 5.3578±0.2000°, 10.7076±0.2000°, 13.3946±0.2000°, 16.0867±0.2000°, and 18.7923±0.2000°.

In some embodiments of the application, the X-ray powder diffraction pattern of the above crystal Form B has characteristic diffraction peaks at the following 2θ angles: 5.3578±0.2000°, 10.7076±0.2000°, 13.3946±0.2000°, 16.0867±0.2000°, 18.7923±0.2000°, and 26.9882±0.2000°.

In some embodiments of the application, the X-ray powder diffraction pattern of the above crystal Form B has characteristic diffraction peaks at the following 2θ angles: 5.3578°, 10.7076°, 13.3946°, 16.0867°, 17.2158°, 18.7923°, 20.0218°, 20.7079°, 21.5030°, 23.0606°, 24.3029°, 25.5466°, 26.9882°, 28.4762°, 29.7465°, 30.8461°, 32.5003°, 35.2977°, 37.0829°, and 38.1654°.

In some embodiments of the application, the XRPD pattern of the above crystal Form B is substantively shown in FIG. 4.

In some embodiments of the application, the analysis data of the XRPD pattern of the above crystal Form B is shown in Table 2:

TABLE 2
Analysis data of the XRPD pattern for
crystal Form B of formula (II) compound

| | 2θ | Interplanar | Relative |
|---|---|---|---|
| No. | angle (°) | spacing (Å) | intensity (%) |
| 1 | 5.3578 | 16.49 | 22.41 |
| 2 | 10.7076 | 8.26 | 35.87 |
| 3 | 13.3946 | 6.61 | 100.00 |
| 4 | 16.0867 | 5.51 | 77.32 |
| 5 | 17.2158 | 5.15 | 0.57 |
| 6 | 18.7923 | 4.72 | 95.60 |
| 7 | 20.0218 | 4.43 | 1.07 |
| 8 | 20.7079 | 4.29 | 0.46 |
| 9 | 21.5030 | 4.13 | 2.74 |
| 10 | 23.0606 | 3.86 | 0.69 |
| 11 | 24.3029 | 3.66 | 0.55 |
| 12 | 25.5466 | 3.49 | 0.36 |
| 13 | 26.9882 | 3.30 | 10.95 |
| 14 | 28.4762 | 3.13 | 0.16 |
| 15 | 29.7465 | 3.00 | 1.33 |
| 16 | 30.8461 | 2.90 | 0.30 |
| 17 | 32.5003 | 2.76 | 0.57 |
| 18 | 35.2977 | 2.54 | 2.22 |
| 19 | 37.0829 | 2.42 | 0.12 |
| 20 | 38.1654 | 2.36 | 0.76 |

The present application provides crystal Form C of formula (II) compound, characterized in that the X-ray powder diffraction pattern of the crystal Form C has characteristic diffraction peaks at the following 2θ angles: 5.2235±0.2000°, 13.0633±0.2000°, and 18.3202±0.2000°.

In some embodiments of the application, the X-ray powder diffraction pattern of the above crystal Form C has characteristic diffraction peaks at the following 2θ angles: 5.2235±0.2000°, 13.0633±0.2000°, 18.3202±0.2000°, 19.0603±0.2000°, and 20.9687±0.2000°.

In some embodiments of the application, the X-ray powder diffraction pattern of the above crystal Form C has characteristic diffraction peaks at the following 2θ angles: 5.2235±0.2000°, 10.6330±0.2000°, 13.0633±0.2000°, 18.3202±0.2000°, 19.0603±0.2000°, 20.9687±0.2000°, 23.0438±0.2000°, and 23.6332±0.2000°.

In some embodiments of the application, the X-ray powder diffraction pattern of the above crystal Form C has characteristic diffraction peaks at the following 2θ angles: 5.2235°, 10.2060°, 10.4366°, 10.6330°, 11.6394°, 13.0633°, 16.3161°, 16.8840°, 17.9453°, 18.3202°, 19.0603°, 19.6631°, 20.5231°, 20.9687°, 21.3445°, 21.9752°, 22.4037°, 23.0438°, 23.3931°, 23.6332°, 23.9781°, 24.7774°, 25.4626°, 26.2681°, 26.7514°, 28.0533°, 28.9221°, 29.4437°, 30.4397°, 30.9179°, 32.6038°, 34.3656°, 36.3357°, and 37.4691°.

In some embodiments of the application, the XRPD pattern of the above crystal Form C is substantively shown in FIG. 5.

In some embodiments of the application, the analysis data of the XRPD pattern of the above crystal Form C is shown in Table 3:

TABLE 3
Analysis data of the XRPD pattern for
crystal Form C of formula (II) compound

| | 2θ | Interplanar | Relative |
|---|---|---|---|
| No. | angle (°) | spacing (Å) | intensity (%) |
| 1 | 5.2235 | 16.92 | 39.78 |
| 2 | 10.2060 | 8.67 | 9.82 |
| 3 | 10.4366 | 8.48 | 15.73 |
| 4 | 10.6330 | 8.32 | 19.12 |
| 5 | 11.6394 | 7.60 | 1.78 |
| 6 | 13.0633 | 6.78 | 100.00 |
| 7 | 16.3161 | 5.43 | 2.90 |
| 8 | 16.8840 | 5.25 | 3.72 |
| 9 | 17.9453 | 4.94 | 6.69 |
| 10 | 18.3202 | 4.84 | 37.23 |
| 11 | 19.0603 | 4.66 | 28.82 |
| 12 | 19.6631 | 4.51 | 4.48 |
| 13 | 20.5231 | 4.33 | 19.22 |
| 14 | 20.9687 | 4.24 | 33.37 |
| 15 | 21.3445 | 4.16 | 11.79 |
| 16 | 21.9752 | 4.04 | 4.98 |
| 17 | 22.4037 | 3.97 | 4.49 |
| 18 | 23.0438 | 3.86 | 24.30 |
| 19 | 23.3931 | 3.80 | 15.53 |
| 20 | 23.6332 | 3.76 | 25.49 |
| 21 | 23.9781 | 3.71 | 8.46 |
| 22 | 24.7774 | 3.59 | 8.95 |
| 23 | 25.4626 | 3.50 | 7.13 |
| 24 | 26.2681 | 3.39 | 5.04 |
| 25 | 26.7514 | 3.33 | 3.12 |
| 26 | 28.0533 | 3.18 | 2.12 |
| 27 | 28.9221 | 3.09 | 2.23 |
| 28 | 29.4437 | 3.03 | 1.06 |
| 29 | 30.4397 | 2.94 | 2.13 |
| 30 | 30.9179 | 2.89 | 2.24 |
| 31 | 32.6038 | 2.75 | 1.47 |
| 32 | 34.3656 | 2.61 | 1.81 |
| 33 | 36.3357 | 2.47 | 1.62 |
| 34 | 37.4691 | 2.40 | 0.52 |

The present application provides crystal Form D of formula (II) compound, characterized in that the X-ray powder diffraction pattern of the crystal Form D has characteristic diffraction peaks at the following 2θ angles: 5.4120±0.2000°, 13.5502±0.2000°, and 24.5140±0.2000°.

In some embodiments of the application, the X-ray powder diffraction pattern of the above crystal Form D has characteristic diffraction peaks at the following 2θ angles: 5.4120±0.2000°, 8.1209±0.2000°, 13.5502±0.2000°, 16.2767±0.2000°, and 24.5140±0.2000°.

In some embodiments of the application, the X-ray powder diffraction pattern of the above crystal Form D has characteristic diffraction peaks at the following 2θ angles: 5.4120±0.2000°, 8.1209±0.2000°, 13.5502±0.2000°, 16.2767±0.2000°, 18.9363±0.2000°, 20.4857±0.2000°, 21.7578±0.2000°, and 24.5140±0.2000°.

In some embodiments of the application, the X-ray powder diffraction pattern of the above crystal Form D has characteristic diffraction peaks at the following 2θ angles: 5.4120°, 8.1209°, 10.4811°, 10.8250°, 11.4035°, 12.8266°, 13.5502°, 14.6390°, 16.2767°, 17.3099°, 17.6879°, 18.9363°, 20.4857°, 20.9317°, 21.4104°, 21.7578°, 21.9895°, 22.3592°, 22.9155°, 23.6785°, 24.5140°, 25.0791°, 25.8055°, 26.2813°, 27.7237°, 29.5078°, 30.9747°, 36.0140°, and 36.8748°.

In some embodiments of the application, the XRPD pattern of the above crystal Form D is substantively shown in FIG. 6.

In some embodiments of the application, the analysis data of the XRPD pattern of the above crystal Form D is shown in Table 4:

TABLE 4
Analysis data of the XRPD pattern for
crystal Form D of formula (II) compound

| | 2θ | Interplanar | Relative |
|---|---|---|---|
| No. | angle (°) | spacing (Å) | intensity (%) |
| 1 | 5.4120 | 16.33 | 100.00 |
| 2 | 8.1209 | 10.89 | 16.57 |
| 3 | 10.4811 | 8.44 | 8.86 |
| 4 | 10.8250 | 8.17 | 1.97 |
| 5 | 11.4035 | 7.76 | 3.88 |
| 6 | 12.8266 | 6.90 | 2.62 |
| 7 | 13.5502 | 6.53 | 22.17 |
| 8 | 14.6390 | 6.05 | 4.16 |
| 9 | 16.2767 | 5.45 | 18.32 |
| 10 | 17.3099 | 5.12 | 1.29 |
| 11 | 17.6879 | 5.01 | 1.28 |
| 12 | 18.9363 | 4.69 | 12.07 |
| 13 | 20.4857 | 4.34 | 9.02 |
| 14 | 20.9317 | 4.24 | 3.33 |
| 15 | 21.4104 | 4.15 | 6.48 |
| 16 | 21.7578 | 4.08 | 16.17 |
| 17 | 21.9895 | 4.04 | 3.80 |
| 18 | 22.3592 | 3.98 | 2.60 |
| 19 | 22.9155 | 3.88 | 0.69 |
| 20 | 23.6785 | 3.76 | 1.40 |
| 21 | 24.5140 | 3.63 | 21.64 |
| 22 | 25.0791 | 3.55 | 1.33 |
| 23 | 25.8055 | 3.45 | 3.77 |
| 24 | 26.2813 | 3.39 | 2.53 |
| 25 | 27.7237 | 3.22 | 0.64 |
| 26 | 29.5078 | 3.03 | 0.70 |
| 27 | 30.9747 | 2.89 | 0.28 |
| 28 | 36.0140 | 2.49 | 0.85 |
| 29 | 36.8748 | 2.44 | 0.71 |

The present application provides crystal Form E of formula (II) compound, characterized in that the X-ray powder diffraction pattern of the crystal Form E has characteristic diffraction peaks at the following 2θ angles: 18.8216±0.2000°, 20.0568±0.2000°, and 25.1936±0.2000°.

In some embodiments of the application, the X-ray powder diffraction pattern of the above crystal Form E has characteristic diffraction peaks at the following 2θ angles: 10.8415±0.2000°, 13.3963±0.2000°, 18.8216±0.2000°, 20.0568±0.2000°, and 25.1936±0.2000°.

In some embodiments of the application, the X-ray powder diffraction pattern of the above crystal Form E has characteristic diffraction peaks at the following 2θ angles: 10.3742±0.2000°, 10.8415±0.2000°, 13.3963±0.2000°, 16.1259±0.2000°, 18.8216±0.2000°, 20.0568±0.2000°, 20.7958±0.2000°, and 25.1936±0.2000°.

The present application provides crystal Form E of formula (II) compound, characterized in that the X-ray powder diffraction pattern of the crystal Form E has characteristic diffraction peaks at the following 2θ angles: 18.8216±0.2000°, 20.0568±0.2000°, and/or 25.1936±0.2000°, and/or 10.8415±0.2000°, and/or 13.3963±0.2000°, and/or 10.3742±0.2000°, and/or 16.1259±0.20000, and/or 20.7958±0.2000°, and/or 5.3759±0.2000°, and/or 11.5008±0.2000°, and/or 15.7418±0.2000°, and/or 16.6392±0.2000°, and/or 17.2881±0.2000°, and/or 19.2577±0.2000°, and/or 21.4850±0.2000°, and/or 22.1639±0.2000°, and/or 23.0777±0.2000°, and/or 24.1467±0.2000°, and/or 24.6828±0.2000°, and/or 25.5960±0.2000°, and/or 26.2392±0.2000°, and/or 26.7365±0.2000°, and/or 27.1146±0.2000°, and/or 27.8176±0.2000°, and/or 28.5679±0.2000°, and/or 29.6443±0.2000°, and/or 30.8753±0.2000°, and/or 33.0123±0.2000°, and/or 33.8173±0.2000°, and/or 36.5754±0.2000°.

In some embodiments of the application, the X-ray powder diffraction pattern of the above crystal Form E has characteristic diffraction peaks at the following 2θ angles: 5.3759°, 10.3742°, 10.8415°, 11.5008°, 13.3963°, 15.7418°, 16.1259°, 16.6392°, 17.2881°, 18.8216°, 19.2577°, 20.0568°, 20.7958°, 21.4850°, 22.1639°, 23.0777°, 24.1467°, 24.6828°, 25.1936°, 25.5960°, 26.2392°, 26.7365°, 27.1146°, 27.8176°, 28.5679°, 29.6443°, 30.8753°, 33.0123°, 33.8173°, and 36.57540.

In some embodiments of the application, the XRPD pattern of the above crystal Form E is substantively shown in FIG. 7.

In some embodiments of the application, the analysis data of the XRPD pattern of the above crystal Form E is shown in Table 5:

TABLE 5
Analysis data of the XRPD pattern for
crystal Form E of formula (II) compound

| | 2θ | Interplanar | Relative |
|---|---|---|---|
| No. | angle (°) | spacing (Å) | intensity (%) |
| 1 | 5.3759 | 16.44 | 31.42 |
| 2 | 10.3742 | 8.53 | 56.51 |
| 3 | 10.8415 | 8.16 | 61.26 |
| 4 | 11.5008 | 7.69 | 12.42 |
| 5 | 13.3963 | 6.61 | 57.86 |
| 6 | 15.7418 | 5.63 | 34.77 |
| 7 | 16.1259 | 5.50 | 44.38 |
| 8 | 16.6392 | 5.33 | 18.46 |
| 9 | 17.2881 | 5.13 | 13.03 |
| 10 | 18.8216 | 4.71 | 72.80 |
| 11 | 19.2577 | 4.61 | 29.83 |
| 12 | 20.0568 | 4.43 | 100.00 |
| 13 | 20.7958 | 4.27 | 47.74 |
| 14 | 21.4850 | 4.14 | 32.22 |
| 15 | 22.1639 | 4.01 | 35.12 |
| 16 | 23.0777 | 3.85 | 30.63 |
| 17 | 24.1467 | 3.69 | 11.91 |
| 18 | 24.6828 | 3.61 | 14.62 |
| 19 | 25.1936 | 3.53 | 68.15 |
| 20 | 25.5960 | 3.48 | 66.21 |
| 21 | 26.2392 | 3.40 | 40.58 |
| 22 | 26.7365 | 3.33 | 25.79 |
| 23 | 27.1146 | 3.29 | 24.80 |
| 24 | 27.8176 | 3.21 | 11.15 |
| 25 | 28.5679 | 3.12 | 22.26 |
| 26 | 29.6443 | 3.01 | 9.99 |
| 27 | 30.8753 | 2.90 | 20.07 |
| 28 | 33.0123 | 2.71 | 9.98 |
| 29 | 33.8173 | 2.65 | 7.88 |
| 30 | 36.5754 | 2.46 | 5.11 |

In some embodiments of the application, the differential scanning calorimetric curve of the above crystal Form E has a peak value of endothermic peak at 91.0±3.0° C. and 149.9±3.0° C., respectively.

In some embodiments of the application, the DSC thermogram of the crystal Form E is substantively shown in FIG. 8.

In some embodiments of the application, the thermogravimetric analysis curve of the crystal Form E has a weight loss of 6.37% at 150.0±3.0° C.

In some embodiments of the application, the TGA spectrum of the crystal Form E is substantively shown in FIG. 9.

The present application provides crystal Form F of formula (II) compound, characterized in that the X-ray powder diffraction pattern of the crystal Form F has characteristic diffraction peaks at the following 2θ angles: 5.4518±0.2000°, 19.3372±0.2000°, and 20.6984±0.2000°.

In some embodiments of the application, the X-ray powder diffraction pattern of the above crystal Form F has characteristic diffraction peaks at the following 2θ angles: 5.4518±0.2000°, 10.5789±0.2000°, 16.4789±0.2000°, 19.3372±0.2000°, and 20.6984±0.2000°.

In some embodiments of the application, the X-ray powder diffraction pattern of the above crystal Form F has characteristic diffraction peaks at the following 2θ angles: 5.4518±0.2000°, 10.5789±0.2000°, 16.4789±0.2000°, 17.7935±0.2000°, 19.3372±0.2000°, 20.6984±0.2000°, 22.3282±0.2000°, and 26.3477±0.2000°.

In some embodiments of the application, the X-ray powder diffraction pattern of the above crystal Form F has characteristic diffraction peaks at the following 2θ angles: 5.4518°, 10.5789°, 11.5837°, 13.0825°, 13.6602°, 14.9423°, 16.4789°, 17.7935°, 19.3372°, 20.6984°, 21.2538°, 22.3282°, 24.7246°, 26.3477°, 27.4475°, and 28.7110°.

In some embodiments of the application, the XRPD pattern of the above crystal Form F is substantively shown in FIG. 10.

In some embodiments of the application, the analysis data of the XRPD pattern of the above crystal Form F is shown in Table 6:

TABLE 6
Analysis data of the XRPD pattern for
crystal Form F of formula (II) compound

| | 2θ | Interplanar | Relative |
|---|---|---|---|
| No. | angle (°) | spacing (Å) | intensity (%) |
| 1 | 5.4518 | 16.21 | 100.00 |
| 2 | 10.5789 | 8.36 | 56.39 |
| 3 | 11.5837 | 7.64 | 19.53 |
| 4 | 13.0825 | 6.77 | 24.26 |
| 5 | 13.6602 | 6.48 | 21.72 |
| 6 | 14.9423 | 5.93 | 22.73 |
| 7 | 16.4789 | 5.38 | 42.04 |
| 8 | 17.7935 | 4.98 | 29.67 |
| 9 | 19.3372 | 4.59 | 65.38 |
| 10 | 20.6984 | 4.29 | 76.94 |
| 11 | 21.2538 | 4.18 | 33.28 |
| 12 | 22.3282 | 3.98 | 34.91 |
| 13 | 24.7246 | 3.60 | 23.24 |
| 14 | 26.3477 | 3.38 | 26.72 |
| 15 | 27.4475 | 3.25 | 10.56 |
| 16 | 28.7110 | 3.11 | 7.42 |

The present application provides crystal Form G of formula (II) compound, characterized in that the X-ray powder diffraction pattern of the crystal Form G has characteristic diffraction peaks at the following 2θ angles: 13.2453±0.2000°, 18.1001±0.2000°, and 20.5915±0.2000°.

In some embodiments of the application, the X-ray powder diffraction pattern of the above crystal Form G has characteristic diffraction peaks at the following 2θ angles: 5.3356±0.2000°, 13.2453±0.2000°, 18.1001±0.2000°, 19.3293±0.2000°, and 20.5915±0.2000°.

In some embodiments of the application, the X-ray powder diffraction pattern of the above crystal Form G has characteristic diffraction peaks at the following 2θ angles: 5.3356±0.2000°, 13.2453±0.2000°, 18.1001±0.2000°, 18.5528±0.2000°, 19.3293±0.2000°, 19.7622±0.2000°, 20.5915±0.2000°, and 23.5540±0.2000°.

In some embodiments of the application, the X-ray powder diffraction pattern of the above crystal Form G has characteristic diffraction peaks at the following 2θ angles: 5.3356°, 10.7072°, 13.2453°, 14.9037°, 16.6883°, 18.1001°, 18.5528°, 19.3293°, 19.7622°, 20.5915°, 21.3864°, 22.0249°, 22.5324°, 23.1856°, 23.5540°, 24.9841°, 25.5102°, 26.9502°, 28.1389°, 29.0651°, and 30.3926°.

In some embodiments of the application, the XRPD pattern of the above crystal Form G is substantively shown in FIG. 11.

In some embodiments of the application, the analysis data of the XRPD pattern of the above crystal Form G is shown in Table 7:

TABLE 7
Analysis data of the XRPD pattern for
crystal Form G of formula (II) compound

| | 2θ | Interplanar | Relative |
|---|---|---|---|
| No. | angle (°) | spacing (Å) | intensity (%) |
| 1 | 5.3356 | 16.56 | 38.92 |
| 2 | 10.7072 | 8.26 | 31.26 |
| 3 | 13.2453 | 6.68 | 81.85 |
| 4 | 14.9037 | 5.94 | 12.34 |
| 5 | 16.6883 | 5.31 | 20.65 |
| 6 | 18.1001 | 4.90 | 49.33 |
| 7 | 18.5528 | 4.78 | 38.63 |
| 8 | 19.3293 | 4.59 | 42.12 |
| 9 | 19.7622 | 4.49 | 34.92 |
| 10 | 20.5915 | 4.31 | 100.00 |
| 11 | 21.3864 | 4.15 | 28.61 |
| 12 | 22.0249 | 4.04 | 28.11 |
| 13 | 22.5324 | 3.95 | 27.37 |
| 14 | 23.1856 | 3.84 | 32.46 |
| 15 | 23.5540 | 3.78 | 36.17 |
| 16 | 24.9841 | 3.56 | 18.59 |
| 17 | 25.5102 | 3.49 | 25.78 |
| 18 | 26.9502 | 3.31 | 14.68 |
| 19 | 28.1389 | 3.17 | 16.48 |
| 20 | 29.0651 | 3.07 | 11.85 |
| 21 | 30.3926 | 2.94 | 14.71 |

The present application provides crystal Form H of formula (II) compound, characterized in that the X-ray powder diffraction pattern of the crystal Form H has characteristic diffraction peaks at the following 2θ angles: 5.2142±0.2000°, 9.2475±0.2000°, and 17.8933±0.2000°.

In some embodiments of the application, the X-ray powder diffraction pattern of the above crystal Form H has characteristic diffraction peaks at the following 2θ angles: 5.2142±0.2000°, 9.2475±0.2000°, 17.8933±0.2000°, 22.0544±0.2000°, and 23.0224±0.2000°.

In some embodiments of the application, the X-ray powder diffraction pattern of the above crystal Form H has characteristic diffraction peaks at the following 2θ angles: 5.2142±0.2000°, 9.2475±0.2000°, 17.8933±0.2000°, 19.3686±0.2000°, 20.2031±0.2000°, 21.2391±0.2000°, 22.0544±0.2000°, and 23.0224±0.2000°.

In some embodiments of the application, the X-ray powder diffraction pattern of the above crystal Form H has characteristic diffraction peaks at the following 2θ angles: 5.2142°, 9.2475°, 10.2712°, 13.6133°, 15.5872°, 16.1306°, 17.8933°, 18.7847°, 19.3686°, 20.2031°, 20.7049°, 21.2391°, 22.0544°, 23.0224°, 24.1930°, and 26.9749°.

In some embodiments of the application, the XRPD pattern of the above crystal Form H is substantively shown in FIG. 12.

In some embodiments of the application, the analysis data of the XRPD pattern of the above crystal Form H is shown in Table 8:

TABLE 8
Analysis data of the XRPD pattern for
crystal Form H of formula (II) compound

| | 2θ | Interplanar | Relative |
|---|---|---|---|
| No. | angle (°) | spacing (Å) | intensity (%) |
| 1 | 5.2142 | 16.95 | 77.62 |
| 2 | 9.2475 | 9.56 | 100.00 |
| 3 | 10.2712 | 8.61 | 19.75 |
| 4 | 13.6133 | 6.50 | 24.36 |
| 5 | 15.5872 | 5.69 | 13.48 |
| 6 | 16.1306 | 5.49 | 10.61 |
| 7 | 17.8933 | 4.96 | 98.95 |
| 8 | 18.7847 | 4.72 | 33.98 |
| 9 | 19.3686 | 4.58 | 50.03 |
| 10 | 20.2031 | 4.40 | 42.56 |
| 11 | 20.7049 | 4.29 | 34.89 |
| 12 | 21.2391 | 4.18 | 46.00 |
| 13 | 22.0544 | 4.03 | 57.00 |
| 14 | 23.0224 | 3.86 | 53.39 |
| 15 | 24.1930 | 3.68 | 14.14 |
| 16 | 26.9749 | 3.31 | 13.11 |

The present application provides crystal Form I of formula (II) compound, characterized in that the X-ray powder diffraction pattern of the crystal Form I has characteristic diffraction peaks at the following 2θ angles: 5.1906±0.2000°, 12.9022±0.2000°, and 15.4944±0.2000°.

In some embodiments of the application, the X-ray powder diffraction pattern of the above crystal Form I has characteristic diffraction peaks at the following 2θ angles: 5.1906±0.2000°, 10.3443±0.2000°, 12.9022±0.2000°, 15.4944±0.2000°, and 18.08160.2000°.

In some embodiments of the application, the X-ray powder diffraction pattern of the above crystal Form I has characteristic diffraction peaks at the following 2θ angles: 5.1906±0.2000°, 10.3443±0.2000°, 12.9022±0.2000°, 13.4027±0.2000°, 15.4944±0.2000°, 16.1068±0.2000°, 18.0816±0.2000°, and 18.8121±0.2000°.

In some embodiments of the application, the X-ray powder diffraction pattern of the above crystal Form I has characteristic diffraction peaks at the following 2θ angles: 5.1906°, 7.7331°, 10.3443°, 10.7143°, 12.9022°, 13.4027°, 15.4944°, 16.1068°, 18.0816°, 18.8121°, 23.3219°, 26.9651°, and 28.5725°.

In some embodiments of the application, the XRPD pattern of the above crystal Form I is substantively shown in FIG. 13.

In some embodiments of the application, the analysis data of the XRPD pattern of the above crystal Form I is shown in Table 9:

TABLE 9
Analysis data of the XRPD pattern for
crystal Form I of formula (II) compound

| | 2θ | Interplanar | Relative |
|---|---|---|---|
| No. | angle (°) | spacing (Å) | intensity (%) |
| 1 | 5.1906 | 17.03 | 76.84 |
| 2 | 7.7331 | 11.43 | 6.90 |
| 3 | 10.3443 | 8.55 | 44.85 |
| 4 | 10.7143 | 8.26 | 19.73 |
| 5 | 12.9022 | 6.86 | 100.00 |
| 6 | 13.4027 | 6.61 | 44.08 |
| 7 | 15.4944 | 5.72 | 59.72 |
| 8 | 16.1068 | 5.50 | 30.34 |
| 9 | 18.0816 | 4.91 | 53.95 |
| 10 | 18.8121 | 4.72 | 32.46 |
| 11 | 23.3219 | 3.81 | 1.87 |
| 12 | 26.9651 | 3.31 | 2.59 |
| 13 | 28.5725 | 3.12 | 1.35 |

The present application further provides formula (III) compound,

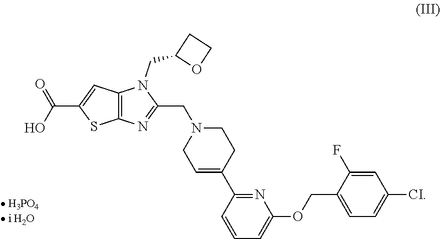

wherein i is selected from 0-2, for example, it may be 0, ½, ⅔, 1, 1.5, or 2.

The present application provides crystal Form J of formula (III) compound, characterized in that the X-ray powder diffraction pattern of the crystal Form J has characteristic diffraction peaks at the following 2θ angles: 6.763±0.200°, 13.619±0.200°, and 18.128±0.200°.

In some embodiments of the application, the X-ray powder diffraction pattern of the above crystal Form J has characteristic diffraction peaks at the following 2θ angles: 6.763±0.200°, 13.619±0.200°, 15.848±0.200°, 18.128±0.200°, and 24.643±0.200°.

In some embodiments of the application, the X-ray powder diffraction pattern of the above crystal Form J has characteristic diffraction peaks at the following 2θ angles: 6.763±0.200°, 13.619±0.200°, 15.848±0.200°, 18.128±0.200°, 19.438±0.200°, 21.422±0.200°, 22.621±0.200°, and 24.643±0.200°.

In some embodiments of the application, the X-ray powder diffraction pattern of the above crystal Form J has characteristic diffraction peaks at the following 2θ angles: 6.763°, 9.065°, 9.535°, 11.902°, 12.066°, 12.874°, 13.619°, 14.169°, 14.918°, 15.848°, 16.105°, 16.573°, 17.620°, 18.128°, 18.510°, 18.826°, 19.107°, 19.438°, 20.538°, 21.191°, 21.422°, 21.794°, 22.416°, 22.621°, 22.806°, 23.376°, 23.609°, 24.643°, 25.149°, 25.650°, 27.329°, 28.990°, 29.368°, 29.916°, 30.162°, 31.416°, 33.040°, 33.684°, 35.327°, and 37.570°.

In some embodiments of the application, the XRPD pattern of the above crystal Form J is substantively shown in FIG. 14.

In some embodiments of the application, the analysis data of the XRPD pattern of the above crystal Form J is shown in Table 10:

TABLE 10
Analysis data of the XRPD pattern for crystal
Form J of formula (III) compound

| | 2θ | Interplanar | Intensity | Relative |
|---|---|---|---|---|
| No. | angle (°) | spacing(Å) | (count) | intensity (%) |
| 1 | 6.763 | 13.05990 | 426.0250 | 31.5 |
| 2 | 9.065 | 9.74724 | 168.5700 | 10.5 |
| 3 | 9.535 | 9.26836 | 61.4809 | 1.6 |
| 4 | 11.902 | 7.42983 | 97.7636 | 4.0 |
| 5 | 12.066 | 7.32918 | 142.8480 | 7.7 |
| 6 | 12.874 | 6.87114 | 107.5660 | 4.1 |
| 7 | 13.619 | 6.49674 | 1233.3700 | 100.0 |
| 8 | 14.169 | 6.24553 | 166.2780 | 9.1 |
| 9 | 14.918 | 5.93385 | 121.2730 | 5.5 |
| 10 | 15.848 | 5.58764 | 371.0470 | 26.6 |
| 11 | 16.105 | 5.49891 | 165.7840 | 9.2 |
| 12 | 16.573 | 5.34480 | 183.2170 | 10.8 |
| 13 | 17.620 | 5.02958 | 142.8340 | 6.8 |
| 14 | 18.128 | 4.88974 | 598.7520 | 45.3 |
| 15 | 18.510 | 4.78949 | 118.5000 | 4.1 |
| 16 | 18.826 | 4.70987 | 132.4340 | 5.2 |
| 17 | 19.107 | 4.64124 | 182.1470 | 9.4 |
| 18 | 19.438 | 4.56298 | 219.1620 | 12.6 |
| 19 | 20.538 | 4.32108 | 172.9960 | 8.4 |
| 20 | 21.191 | 4.18933 | 251.4230 | 14.2 |
| 21 | 21.422 | 4.14469 | 355.4930 | 22.8 |
| 22 | 21.794 | 4.07463 | 187.8470 | 8.1 |
| 23 | 22.416 | 3.96300 | 179.6320 | 7.0 |
| 24 | 22.621 | 3.92752 | 229.4550 | 11.2 |
| 25 | 22.806 | 3.89611 | 208.0040 | 9.3 |
| 26 | 23.376 | 3.80238 | 204.9310 | 9.0 |
| 27 | 23.609 | 3.76543 | 159.3610 | 5.1 |
| 28 | 24.643 | 3.60968 | 405.4030 | 26.5 |
| 29 | 25.149 | 3.53817 | 164.7130 | 6.3 |
| 30 | 25.650 | 3.47019 | 127.1890 | 3.6 |
| 31 | 27.329 | 3.26072 | 88.6437 | 2.0 |
| 32 | 28.990 | 3.07753 | 122.5600 | 5.3 |
| 33 | 29.368 | 3.03878 | 81.8571 | 1.9 |
| 34 | 29.916 | 2.98438 | 112.9290 | 4.8 |
| 35 | 30.162 | 2.96058 | 94.5396 | 3.4 |
| 36 | 31.416 | 2.84519 | 63.8882 | 1.5 |
| 37 | 33.040 | 2.70895 | 97.6047 | 4.1 |
| 38 | 33.684 | 2.65864 | 68.4512 | 2.0 |
| 39 | 35.327 | 2.53870 | 59.5384 | 1.7 |
| 40 | 37.570 | 2.39212 | 66.5015 | 2.5 |

The present application further provides formula (IV) compound,

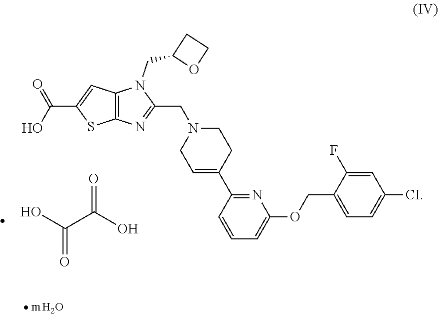

wherein m is selected from 0-2, for example, it may be 0, ½, ⅔, 1, 1.5, or 2.

In some embodiments of the application, the formula (IV) compound has the following structures:

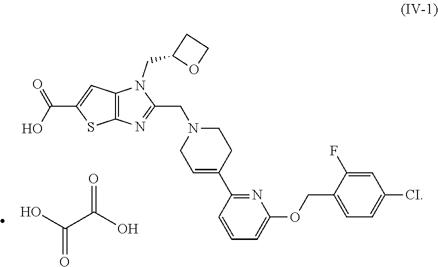

The present application provides crystal Form K of formula (IV-1) compound, characterized in that the X-ray powder diffraction pattern of the crystal Form K has characteristic diffraction peaks at the following 2θ angles: 16.860±0.200°, 18.189±0.200°, and 20.709±0.200°.

In some embodiments of the application, the X-ray powder diffraction pattern of the above crystal Form K has characteristic diffraction peaks at the following 2θ angles: 9.972±0.200°, 16.860±0.200°, 18.189±0.200°, 20.709±0.200°, and 23.950±0.200°.

In some embodiments of the application, the X-ray powder diffraction pattern of the above crystal Form K has characteristic diffraction peaks at the following 2θ angles: 4.581±0.200°, 9.972±0.200°, 10.392±0.200°, 16.860±0.200°, 18.189±0.200°, 20.709±0.200°, 23.950±0.200°, and 26.841±0.200°.

In some embodiments of the application, the X-ray powder diffraction pattern of the above crystal Form K has characteristic diffraction peaks at the following 2θ angles: 4.581°, 6.439°, 9.972°, 10.232°, 10.392°, 13.328°, 14.053°, 14.696°, 16.860°, 18.189°, 20.709°, 22.766°, 23.9500, 25.785°, and 26.841°.

In some embodiments of the application, the XRPD pattern of the above crystal Form K is substantively shown in FIG. 15.

In some embodiments of the application, the analysis data of the XRPD pattern of the above crystal Form K is shown in Table 11:

TABLE 11
Analysis data of the XRPD pattern for crystal
Form K of formula (IV-1) compound

| | 2θ | Interplanar | Intensity | Relative |
|---|---|---|---|---|
| No. | angle (°) | spacing(Å) | (count) | intensity (%) |
| 1 | 4.581 | 19.27239 | 211.2910 | 28.1 |
| 2 | 6.439 | 13.71585 | 129.8420 | 16.9 |
| 3 | 9.972 | 8.86296 | 220.0000 | 39.1 |
| 4 | 10.232 | 8.63817 | 207.1560 | 35.9 |
| 5 | 10.392 | 8.50574 | 219.6360 | 38.8 |
| 6 | 13.328 | 6.63803 | 159.6750 | 21.2 |
| 7 | 14.053 | 6.29681 | 175.5610 | 22.4 |
| 8 | 14.696 | 6.02308 | 160.3510 | 17.0 |
| 9 | 16.860 | 5.25431 | 460.9880 | 80.9 |
| 10 | 18.189 | 4.87340 | 561.3880 | 100.0 |
| 11 | 20.709 | 4.28559 | 421.4340 | 62.8 |
| 12 | 22.766 | 3.90297 | 194.6380 | 10.9 |
| 13 | 23.950 | 3.71259 | 309.4320 | 41.6 |
| 14 | 25.785 | 3.45238 | 221.1160 | 23.5 |
| 15 | 26.841 | 3.31883 | 268.7680 | 36.4 |

The present application provides crystal Form L of formula (IV) compound, characterized in that the X-ray powder diffraction pattern of the crystal Form L has characteristic diffraction peaks at the following 2θ angles: 18.726±0.200°, 21.044±0.200°, and 24.648±0.200°.

In some embodiments of the application, the X-ray powder diffraction pattern of the above crystal Form L has characteristic diffraction peaks at the following 2θ angles: 6.494±0.200°, 18.726±0.200°, 19.547±0.200°, 21.044±0.200°, and 24.648±0.200°.

In some embodiments of the application, the X-ray powder diffraction pattern of the above crystal Form L has characteristic diffraction peaks at the following 2θ angles: 6.494±0.200°, 15.366±0.200°, 17.885±0.200°, 18.726±0.200°, 19.547±0.200°, 21.044±0.200°, 24.648±0.200°, and 27.453±0.200°.

In some embodiments of the application, the X-ray powder diffraction pattern of the above crystal Form L has characteristic diffraction peaks at the following 2θ angles: 5.797°, 6.494°, 7.655°, 10.474°, 13.036°, 13.388°, 15.366°, 16.235°, 17.885°, 18.726°, 19.547°, 21.044°, 24.368°, 24.648°, and 27.453°.

In some embodiments of the application, the XRPD pattern of the above crystal Form L is substantively shown in FIG. 16.

In some embodiments of the application, the analysis data of the XRPD pattern of the above crystal Form L is shown in Table 12:

TABLE 12
Analysis data of the XRPD pattern for crystal
Form L of formula (IV) compound

| | 2θ | Interplanar | Intensity | Relative |
|---|---|---|---|---|
| No. | angle (°) | spacing(Å) | (count) | intensity (%) |
| 1 | 5.797 | 15.23338 | 84.7781 | 20.4 |
| 2 | 6.494 | 13.59886 | 117.6260 | 48.3 |
| 3 | 7.655 | 11.53911 | 79.0000 | 23.3 |
| 4 | 10.474 | 8.43907 | 75.1471 | 23.3 |
| 5 | 13.036 | 6.78600 | 75.6188 | 20.7 |
| 6 | 13.388 | 6.60817 | 86.7207 | 28.0 |
| 7 | 15.366 | 5.76165 | 108.1480 | 39.7 |
| 8 | 16.235 | 5.45511 | 81.4001 | 18.4 |
| 9 | 17.885 | 4.95562 | 108.0170 | 32.9 |
| 10 | 18.726 | 4.73493 | 204.6810 | 100.0 |
| 11 | 19.547 | 4.53771 | 170.9440 | 71.6 |
| 12 | 21.044 | 4.21812 | 206.1620 | 95.7 |
| 13 | 24.368 | 3.64980 | 109.0690 | 33.3 |
| 14 | 24.648 | 3.60897 | 164.0680 | 73.9 |
| 15 | 27.453 | 3.24626 | 115.6630 | 40.3 |

The present application further provides formula (V) compound,

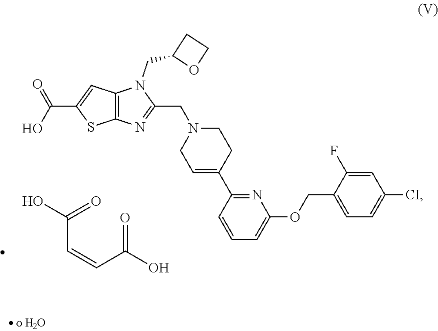

wherein o is selected from 0-2, for example, it may be 0, ½, ⅔, 1, 1.5, or 2.

The present application provides crystal Form M of formula (V) compound, characterized in that the X-ray powder diffraction pattern of the crystal Form M has characteristic diffraction peaks at the following 2θ angles: 5.682±0.200°, 11.389±0.200°, and 17.202±0.200°.

In some embodiments of the application, the X-ray powder diffraction pattern of the above crystal Form M has characteristic diffraction peaks at the following 2θ angles: 5.682±0.200°, 11.389±0.200°, 16.009±0.200°, 17.202±0.200°, and 29.418±0.200°.

In some embodiments of the application, the X-ray powder diffraction pattern of the above crystal Form M has characteristic diffraction peaks at the following 2θ angles: 5.682±0.200°, 11.389±0.200°, 16.009±0.200°, 17.202±0.200°, 17.854±0.200°, 24.147±0.200°, 25.302±0.200°, and 29.418±0.200°.

In some embodiments of the application, the X-ray powder diffraction pattern of the above crystal Form M has characteristic diffraction peaks at the following 2θ angles: 5.682°, 10.619°, 11.389°, 12.280°, 12.637°, 14.434°, 15.764°, 16.009°, 17.202°, 17.854°, 18.783°, 19.352°, 20.673°, 21.044°, 21.729°, 22.632°, 23.341°, 24.147°, 25.302°, 26.982°, 28.950°, 29.418°, 30.123°, and 33.086°.

In some embodiments of the application, the XRPD pattern of the above crystal Form M is substantively shown in FIG. 17.

In some embodiments of the application, the analysis data of the XRPD pattern of the above crystal Form M is shown in Table 13:

TABLE 13
Analysis data of the XRPD pattern for crystal
Form M of formula (V) compound

| | 2θ | Interplanar | Intensity | Relative |
|---|---|---|---|---|
| No. | angle (°) | spacing(Å) | (count) | intensity (%) |
| 1 | 5.682 | 15.54248 | 670.9000 | 100.0 |
| 2 | 10.619 | 8.32400 | 87.0000 | 6.7 |
| 3 | 11.389 | 7.76333 | 448.2610 | 66.8 |
| 4 | 12.280 | 7.20178 | 71.5244 | 3.8 |
| 5 | 12.637 | 6.99921 | 92.0000 | 7.5 |
| 6 | 14.434 | 6.13174 | 100.3550 | 8.5 |
| 7 | 15.764 | 5.61731 | 277.4270 | 36.1 |
| 8 | 16.009 | 5.53179 | 363.2260 | 50.1 |
| 9 | 17.202 | 5.15073 | 631.2960 | 94.0 |
| 10 | 17.854 | 4.96417 | 181.1390 | 18.5 |
| 11 | 18.783 | 4.72048 | 145.9900 | 13.1 |
| 12 | 19.352 | 4.58294 | 110.0720 | 7.8 |
| 13 | 20.673 | 4.29297 | 124.1850 | 11.2 |
| 14 | 21.044 | 4.21813 | 163.4260 | 17.8 |
| 15 | 21.729 | 4.08677 | 92.1371 | 6.1 |
| 16 | 22.632 | 3.92572 | 95.3605 | 5.6 |
| 17 | 23.341 | 3.80810 | 163.7800 | 15.7 |
| 18 | 24.147 | 3.68268 | 230.7000 | 26.0 |
| 19 | 25.302 | 3.51718 | 191.6670 | 19.1 |
| 20 | 26.982 | 3.30185 | 109.2230 | 5.9 |
| 21 | 28.950 | 3.08170 | 120.5480 | 7.8 |
| 22 | 29.418 | 3.03379 | 288.7370 | 36.3 |
| 23 | 30.123 | 2.96437 | 140.2930 | 12.3 |
| 24 | 33.086 | 2.70529 | 74.0298 | 4.1 |

The present application further provides formula (VI) compound,

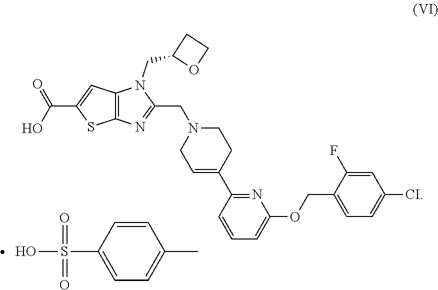

The present application provides crystal Form N of formula (VI) compound, characterized in that the X-ray powder diffraction pattern of the crystal Form N has characteristic diffraction peaks at the following 2θ angles: 11.439±0.200°, 18.363±0.200°, and 28.859±0.200°.

In some embodiments of the application, the X-ray powder diffraction pattern of the above crystal Form N has characteristic diffraction peaks at the following 2θ angles: 11.439±0.200°, 18.363±0.200°, 20.062±0.200°, 22.657±0.200°, and 28.859±0.200°.

In some embodiments of the application, the X-ray powder diffraction pattern of the above crystal Form N has characteristic diffraction peaks at the following 2θ angles: 8.581±0.200°, 11.439±0.200°, 18.363±0.200°, 20.062±0.200°, 22.657±0.200°, 23.664±0.200°, 25.356±0.200°, and 28.859±0.200°.

In some embodiments of the application, the X-ray powder diffraction pattern of the above crystal Form N has characteristic diffraction peaks at the following 2θ angles: 5.697°, 8.581°, 8.801°, 11.439°, 12.051°, 12.568°, 13.226°, 14.357°, 14.846°, 15.655°, 16.120°, 17.223°, 17.880°, 18.363°, 18.914°, 19.236°, 20.062°, 20.397°, 21.454°, 22.657°, 22.991°, 23.664°, 24.391°, 24.844°, 25.356°, 26.003°, 26.573°, 26.956°, 27.309°, 28.283°, 28.859°, 29.089°, 29.935°, 31.553°, 31.847°, 32.174°, 32.715°, 34.782°, and 38.569°.

In some embodiments of the application, the XRPD pattern of the above crystal Form N is substantively shown in FIG. 18.

In some embodiments of the application, the analysis data of the XRPD pattern of the above crystal Form N is shown in Table 14:

TABLE 14
Analysis data of the XRPD pattern for crystal
Form N of formula (VI) compound

| | 2θ | Interplanar | Intensity | Relative |
|---|---|---|---|---|
| No. | angle (°) | spacing(Å) | (count) | intensity (%) |
| 1 | 5.697 | 15.50109 | 89.5945 | 16.8 |
| 2 | 8.581 | 10.29639 | 122.4680 | 44.9 |
| 3 | 8.801 | 10.03905 | 54.7683 | 9.1 |
| 4 | 11.439 | 7.72969 | 223.1990 | 100.0 |
| 5 | 12.051 | 7.33844 | 57.2892 | 11.4 |
| 6 | 12.568 | 7.03732 | 101.1980 | 35.3 |
| 7 | 13.226 | 6.68903 | 65.3537 | 17.5 |
| 8 | 14.357 | 6.16434 | 72.2934 | 20.4 |
| 9 | 14.846 | 5.96222 | 72.5728 | 20.2 |
| 10 | 15.655 | 5.65613 | 76.1569 | 21.5 |
| 11 | 16.120 | 5.49386 | 74.8304 | 20.0 |
| 12 | 17.223 | 5.14447 | 109.4950 | 37.0 |
| 13 | 17.880 | 4.95694 | 58.2274 | 8.8 |
| 14 | 18.363 | 4.82756 | 172.9750 | 69.0 |
| 15 | 18.914 | 4.68815 | 96.0006 | 27.3 |
| 16 | 19.236 | 4.61041 | 83.0672 | 20.3 |
| 17 | 20.062 | 4.42251 | 149.2200 | 56.2 |
| 18 | 20.397 | 4.35043 | 114.6230 | 38.4 |
| 19 | 21.454 | 4.13842 | 67.0976 | 15.6 |
| 20 | 22.657 | 3.92150 | 139.5750 | 53.8 |
| 21 | 22.991 | 3.86523 | 131.1530 | 49.3 |
| 22 | 23.664 | 3.75681 | 116.7200 | 42.2 |
| 23 | 24.391 | 3.64645 | 75.7081 | 19.8 |
| 24 | 24.844 | 3.58098 | 95.3251 | 29.6 |
| 25 | 25.356 | 3.50980 | 123.6270 | 44.3 |
| 26 | 26.003 | 3.42394 | 107.7510 | 35.7 |
| 27 | 26.573 | 3.35170 | 94.1596 | 28.5 |
| 28 | 26.956 | 3.30497 | 57.5013 | 9.4 |
| 29 | 27.309 | 3.26307 | 93.8005 | 29.4 |
| 30 | 28.283 | 3.15288 | 53.0007 | 9.2 |
| 31 | 28.859 | 3.09128 | 214.0480 | 95.2 |
| 32 | 29.089 | 3.06727 | 75.0909 | 21.3 |
| 33 | 29.935 | 2.98253 | 54.5002 | 11.9 |
| 34 | 31.553 | 2.83316 | 49.6036 | 11.6 |
| 35 | 31.847 | 2.80768 | 90.1399 | 32.8 |
| 36 | 32.174 | 2.77986 | 66.5213 | 19.9 |
| 37 | 32.715 | 2.73516 | 61.4286 | 17.1 |
| 38 | 34.782 | 2.57720 | 68.9615 | 20.6 |
| 39 | 38.569 | 2.33239 | 52.9000 | 11.6 |

The present application further provides formula (VII) compound,

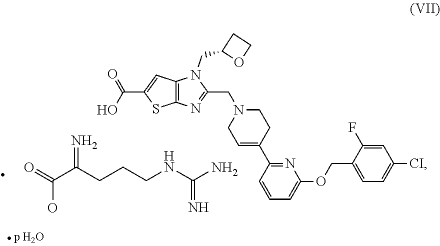

wherein p is selected from 0-3, for example, it may be 0, ½, ⅔, 1, 1.5, 2, 2.5, 2.7, or 3.

The present application provides crystal Form O of formula (VII) compound, characterized in that the X-ray powder diffraction pattern of the crystal Form O has characteristic diffraction peaks at the following 2θ angles: 9.485±0.200°, 11.273±0.200°, and 17.536±0.200°.

In some embodiments of the application, the X-ray powder diffraction pattern of the above crystal Form O has characteristic diffraction peaks at the following 2θ angles: 9.485±0.200°, 11.273±0.200°, 12.495±0.200°, 17.536±0.200°, and 18.874±0.200°.

In some embodiments of the application, the X-ray powder diffraction pattern of the above crystal Form O has characteristic diffraction peaks at the following 2θ angles: 9.485±0.200°, 11.273±0.200°, 12.495±0.200°, 15.005±0.200°, 17.536±0.200°, 185874±0.200°, 19.568±0.200°, and 20.291±0.200°.

In some embodiments of the application, the X-ray powder diffraction pattern of the above crystal Form O has characteristic diffraction peaks at the following 2θ angles: 4.859°, 7.487°, 9.485°, 10.000°, 11.273°, 12.495°, 12.842°, 14.649°, 15.005°, 16.766°, 17.536°, 18.874°, 19.568°, 20.082°, 20.291°, 21.189°, 22.679°, 23.508°, 25.140°, 25.788°, 28.081°, 29.067°, 29.739°, 31.639°, and 35.581°.

In some embodiments of the application, the XRPD pattern of the above crystal Form O is substantively shown in FIG. 19.

In some embodiments of the application, the analysis data of the XRPD pattern of the above crystal Form O is shown in Table 15:

TABLE 15
Analysis data of the XRPD pattern for crystal
Form O of formula (VII) compound

| | 2θ | Interplanar | Intensity | Relative |
|---|---|---|---|---|
| No. | angle (°) | spacing(Å) | (count) | intensity (%) |
| 1 | 4.859 | 18.17302 | 136.8720 | 5.3 |
| 2 | 7.487 | 11.79782 | 91.5449 | 4.4 |
| 3 | 9.485 | 9.31674 | 985.5060 | 96.7 |
| 4 | 10.000 | 8.83820 | 347.5300 | 29.5 |
| 5 | 11.273 | 7.84274 | 1024.1500 | 100.0 |
| 6 | 12.495 | 7.07830 | 574.7970 | 53.7 |
| 7 | 12.842 | 6.88813 | 129.1140 | 7.4 |
| 8 | 14.649 | 6.04194 | 243.2650 | 19.7 |
| 9 | 15.005 | 5.89944 | 362.3520 | 32.1 |
| 10 | 16.766 | 5.28368 | 114.2170 | 4.2 |
| 11 | 17.536 | 5.05323 | 765.8320 | 71.3 |
| 12 | 18.874 | 4.69802 | 744.2180 | 67.4 |
| 13 | 19.568 | 4.53284 | 606.9120 | 52.5 |
| 14 | 20.082 | 4.41801 | 385.0590 | 29.0 |
| 15 | 20.291 | 4.37310 | 452.7620 | 36.0 |
| 16 | 21.189 | 4.18976 | 404.6190 | 31.2 |
| 17 | 22.679 | 3.91775 | 208.9830 | 11.7 |
| 18 | 23.508 | 3.78128 | 167.2290 | 7.8 |
| 19 | 25.140 | 3.53951 | 103.8110 | 2.4 |
| 20 | 25.788 | 3.45200 | 161.5330 | 9.1 |
| 21 | 28.081 | 3.17514 | 101.5000 | 3.6 |
| 22 | 29.067 | 3.06955 | 113.2560 | 4.6 |
| 23 | 29.739 | 3.00173 | 94.5813 | 2.7 |
| 24 | 31.639 | 2.82567 | 93.7721 | 3.4 |
| 25 | 35.581 | 2.52110 | 76.3221 | 2.2 |

The present application provides crystal Form P of formula (VII) compound, characterized in that the X-ray powder diffraction pattern of the crystal Form P has characteristic diffraction peaks at the following 2θ angles: 12.481±0.200°, 14.965±0.200°, and 17.480±0.200°.

In some embodiments of the application, the X-ray powder diffraction pattern of the above crystal Form P has characteristic diffraction peaks at the following 2θ angles: 9.998±0.200°, 12.481±0.200°, 14.965±0.200°, 17.480±0.200°, and 19.969±0.200°.

In some embodiments of the application, the X-ray powder diffraction pattern of the above crystal Form P has characteristic diffraction peaks at the following 2θ angles: 5.007, 7.5120, 9.604°, 9.998°, 12.481°, 14.965°, 17.480°, 18.908°, 19.969°, 20.933°, 25.629°, 27.587°, and 35.3750.

In some embodiments of the application, the XRPD pattern of the above crystal Form P is substantively shown in FIG. 20.

In some embodiments of the application, the analysis data of the XRPD pattern of the above crystal Form P is shown in Table 16:

TABLE 16
Analysis data of the XRPD pattern for crystal
Form P of formula (VII) compound

| | 2θ | Interplanar | Intensity | Relative |
|---|---|---|---|---|
| No. | angle (°) | spacing(Å) | (count) | intensity (%) |
| 1 | 5.007 | 17.63558 | 154.9210 | 4.1 |
| 2 | 7.512 | 11.75886 | 169.6510 | 6.2 |
| 3 | 9.604 | 9.20203 | 176.4390 | 6.3 |
| 4 | 9.998 | 8.83996 | 438.1460 | 20.4 |
| 5 | 12.481 | 7.08630 | 1184.3100 | 60.6 |
| 6 | 14.965 | 5.91540 | 737.2030 | 36.1 |
| 7 | 17.480 | 5.06942 | 1944.7700 | 100.0 |
| 8 | 18.908 | 4.68965 | 291.2900 | 7.5 |
| 9 | 19.969 | 4.44271 | 707.7450 | 29.8 |
| 10 | 20.933 | 4.24037 | 225.1970 | 3.3 |
| 11 | 25.629 | 3.47307 | 231.4040 | 4.4 |
| 12 | 27.587 | 3.23076 | 223.8710 | 5.1 |
| 13 | 35.375 | 2.53535 | 136.9050 | 2.6 |

The present application further provides formula (VIII) compound,

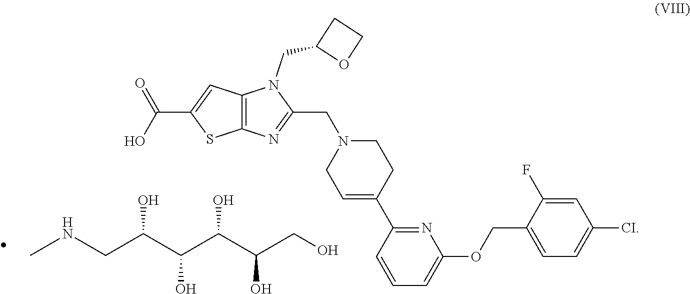

The present application provides crystal Form Q of formula (VIII) compound, characterized in that the X-ray powder diffraction pattern of the crystal Form Q has characteristic diffraction peaks at the following 2θ angles: 6.123±0.200°, 9.130±0.200°, and 12.123±0.200°.

In some embodiments of the application, the X-ray powder diffraction pattern of the above crystal Form Q has characteristic diffraction peaks at the following 2θ angles: 6.123±0.200°, 9.130±0.200°, 12.123±0.200°, 20.095±0.200°, and 22.942±0.200°.

In some embodiments of the application, the X-ray powder diffraction pattern of the above crystal Form Q has characteristic diffraction peaks at the following 2θ angles: 6.123±0.200°, 9.130±0.200°, 12.123±0.200°, 15.071±0.200°, 18.151±0.200°, 20.095±0.200°, 22.045±0.200°, and 22.942±0.200°.

In some embodiments of the application, the X-ray powder diffraction pattern of the above crystal Form Q has characteristic diffraction peaks at the following 2θ angles: 6.123°, 7.677°, 9.130°, 10.013°, 11.598°, 12.123°, 14.770°, 15.071°, 15.986, 16.493°, 16.692°, 17.365°, 18.151°, 18.915°, 20.095°, 20.569°, 21.147°, 22.045°, 22.942°, 24.106°, 24.617°, 26.108°, 27.014°, 27.822°, 28.433°, 29.464°, 30.256°, 30.863°, 31.820°, 334460 34.822°, 36.389°, 37.646°, and 37.8030.

In some embodiments of the application, the XRPD pattern of the above crystal Form Q is substantively shown in FIG. 21.

In some embodiments of the application, the analysis data of the XRPD pattern of the above crystal Form Q is shown in Table 17:

TABLE 17
Analysis data of the XRPD pattern for crystal
Form Q of formula (VIII) compound

| | 2θ | Interplanar | Intensity | Relative |
|---|---|---|---|---|
| No. | angle (°) | spacing(Å) | (count) | intensity (%) |
| 1 | 6.123 | 14.42249 | 907.3920 | 76.4 |
| 2 | 7.677 | 11.50664 | 84.7541 | 2.8 |
| 3 | 9.130 | 9.67823 | 1022.2900 | 86.9 |
| 4 | 10.013 | 8.82668 | 134.3050 | 6.3 |
| 5 | 11.598 | 7.62398 | 122.3030 | 5.5 |
| 6 | 12.123 | 7.29479 | 1161.0700 | 100.0 |
| 7 | 14.770 | 5.99286 | 132.9200 | 6.9 |
| 8 | 15.071 | 5.87383 | 397.0220 | 30.4 |
| 9 | 15.986 | 5.53952 | 231.8630 | 14.4 |
| 10 | 16.493 | 5.37039 | 173.8630 | 8.7 |
| 11 | 16.692 | 5.30677 | 150.3330 | 6.5 |
| 12 | 17.365 | 5.10269 | 111.8660 | 2.9 |
| 13 | 18.151 | 4.88337 | 435.3270 | 32.5 |
| 14 | 18.915 | 4.68785 | 117.4870 | 4.4 |
| 15 | 20.095 | 4.41525 | 454.9470 | 33.8 |
| 16 | 20.569 | 4.31463 | 254.2030 | 14.9 |
| 17 | 21.147 | 4.19782 | 417.1830 | 29.2 |
| 18 | 22.045 | 4.02896 | 440.8910 | 31.1 |
| 19 | 22.942 | 3.87336 | 911.1580 | 74.0 |
| 20 | 24.106 | 3.68892 | 198.0170 | 10.2 |
| 21 | 24.617 | 3.61350 | 196.2860 | 10.6 |
| 22 | 26.108 | 3.41040 | 127.0000 | 5.5 |
| 23 | 27.014 | 3.29808 | 208.2860 | 12.0 |
| 24 | 27.822 | 3.20407 | 110.5740 | 2.7 |
| 25 | 28.433 | 3.13663 | 215.8360 | 12.0 |
| 26 | 29.464 | 3.02913 | 175.6300 | 8.3 |
| 27 | 30.256 | 2.95159 | 115.0600 | 2.8 |
| 28 | 30.863 | 2.89490 | 306.8510 | 20.6 |
| 29 | 31.820 | 2.81003 | 106.0640 | 3.2 |
| 30 | 33.446 | 2.67705 | 113.4510 | 4.7 |
| 31 | 34.822 | 2.57436 | 206.0210 | 13.5 |
| 32 | 36.389 | 2.46696 | 130.7540 | 6.2 |
| 33 | 37.646 | 2.38747 | 167.4840 | 9.1 |
| 34 | 37.803 | 2.37786 | 220.4210 | 13.9 |

The present application further provides formula (I) compound,

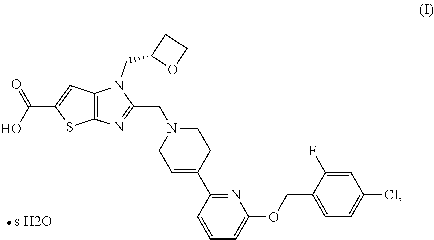

wherein s is selected from 0-3, for example, it may be 0, ½, ⅔, 1, 1.5, 2, 2.5, 2.7, or 3.

In some embodiments of the application, the s is selected from 0, 1, 2 and 3.

In some embodiments of the application, the formula (I) compound has the following structures:

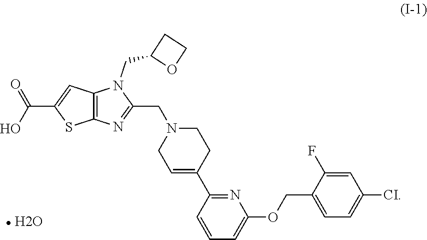

The present application provides crystal Form R of formula (I) compound, characterized in that the X-ray powder diffraction pattern of the crystal Form R has characteristic diffraction peaks at the following 2θ angles: 7.933±0.200°, 15.900±0.200°, and 23.970±0.200°.

In some embodiments of the application, the X-ray powder diffraction pattern of the above crystal Form R has characteristic diffraction peaks at the following 2θ angles: 7.933±0.200°, 11.906±0.200°, 15.900±0.200°, 19.923±0.200°, and 23.970±0.200°.

In some embodiments of the application, the X-ray powder diffraction pattern of the above crystal Form R has characteristic diffraction peaks at the following 2θ angles: 3.935±0.200°, 7.933±0.200°, 11.906±0.200°, 15.900±0.200°, 19.923±0.200°, and 23.970±0.200°.

In some embodiments of the application, the X-ray powder diffraction pattern of the above crystal Form R has characteristic diffraction peaks at the following 2θ angles: 3.935°, 7.933°, 11.906°, 15.900°, 17.731°, 19.923°, 21.294°, 23.503°, 23.970°, 25.040°, 26.811°, 28.041°, 28.913°, 31.180°, 32.172°, 34.091°, and 38.072°.

In some embodiments of the application, the XRPD pattern of the above crystal Form R is substantively shown in FIG. 22.

In some embodiments of the application, the analysis data of the XRPD pattern of the above crystal Form R is shown in Table 18:

TABLE 18
Analysis data of the XRPD pattern for crystal
Form R of formula (I) compound

| | 2θ | Interplanar | Intensity | Relative |
|---|---|---|---|---|
| No. | angle (°) | spacing(Å) | (count) | intensity (%) |
| 1 | 3.935 | 22.43649 | 567.7500 | 14.1 |
| 2 | 7.933 | 11.13559 | 1372.5800 | 41.3 |
| 3 | 11.906 | 7.42732 | 548.6490 | 15.9 |
| 4 | 15.900 | 5.56952 | 3259.2800 | 100.0 |
| 5 | 17.731 | 4.99819 | 117.1610 | 2.1 |
| 6 | 19.923 | 4.45295 | 807.4570 | 23.6 |
| 7 | 21.294 | 4.16917 | 138.4980 | 2.7 |
| 8 | 23.503 | 3.78209 | 236.0010 | 5.3 |
| 9 | 23.970 | 3.70951 | 2521.7100 | 76.5 |
| 10 | 25.040 | 3.55330 | 166.8080 | 3.2 |
| 11 | 26.811 | 3.32249 | 126.9920 | 2.5 |
| 12 | 28.041 | 3.17952 | 237.3540 | 5.8 |
| 13 | 28.913 | 3.08562 | 196.2260 | 4.7 |
| 14 | 31.180 | 2.86626 | 62.3383 | 0.8 |
| 15 | 32.172 | 2.78010 | 56.0000 | 0.7 |
| 16 | 34.091 | 2.62781 | 66.0118 | 1.1 |
| 17 | 38.072 | 2.36170 | 44.6912 | 0.6 |

The present application provides crystal Form S of formula (I) compound, characterized in that the X-ray powder diffraction pattern of the crystal Form S has characteristic diffraction peaks at the following 2θ angles: 20.032±0.200°, 21.239±0.200°, and 23.474±0.200°.

In some embodiments of the application, the X-ray powder diffraction pattern of the above crystal Form S has characteristic diffraction peaks at the following 2θ angles: 15.569±0.200°, 17.781±0.200°, 20.032±0.200°, 21.239±0.200°, and 23.474±0.200°.

In some embodiments of the application, the X-ray powder diffraction pattern of the above crystal Form S has characteristic diffraction peaks at the following 2θ angles: 14.527±0.200°, 15.569±0.200°, 17.781±0.200°, 20.032±0.200°, 21.239±0.200°, 23.474±0.200°, 24.275±0.200°, and 26.863±0.200°.

In some embodiments of the application, the X-ray powder diffraction pattern of the above crystal Form S has characteristic diffraction peaks at the following 2θ angles: 3.871°, 7.901°, 9.387°, 9.859°, 11.461°, 14.527°, 15.569°, 15.906°, 16.743°, 17.781°, 18.461°, 20.032°, 20.3780, 21.239°, 23.474°, 24.275°, 25.367°, 26.863°, 27.063°, 28.954°, 31.135°, and 34.181°.

In some embodiments of the application, the XRPD pattern of the above crystal Form S is substantively shown in FIG. 23.

In some embodiments of the application, the analysis data of the XRPD pattern of the above crystal Form S is shown in Table 19:

TABLE 19
Analysis data of the XRPD pattern for crystal
Form S of formula (I) compound

| | 2θ | Interplanar | Intensity | Relative |
|---|---|---|---|---|
| No. | angle (°) | spacing(Å) | (count) | intensity (%) |
| 1 | 3.871 | 22.80906 | 132.3170 | 10.2 |
| 2 | 7.901 | 11.18113 | 110.6030 | 25.3 |
| 3 | 9.387 | 9.41368 | 77.8009 | 15.1 |
| 4 | 9.859 | 8.96460 | 57.3897 | 8.8 |
| 5 | 11.461 | 7.71439 | 63.3416 | 10.9 |
| 6 | 14.527 | 6.09269 | 124.3410 | 30.9 |
| 7 | 15.569 | 5.68725 | 148.1850 | 38.5 |
| 8 | 15.906 | 5.56743 | 123.0110 | 29.7 |
| 9 | 16.743 | 5.29088 | 62.5341 | 9.3 |
| 10 | 17.781 | 4.98435 | 127.8130 | 31.5 |
| 11 | 18.461 | 4.80220 | 75.4175 | 11.7 |
| 12 | 20.032 | 4.42897 | 199.2930 | 53.6 |
| 13 | 20.378 | 4.35449 | 77.4612 | 11.1 |
| 14 | 21.239 | 4.17987 | 192.5000 | 51.9 |
| 15 | 23.474 | 3.78674 | 327.5390 | 100.0 |
| 16 | 24.275 | 3.66352 | 120.2510 | 28.0 |
| 17 | 25.367 | 3.50829 | 87.1330 | 18.1 |
| 18 | 26.863 | 3.31625 | 108.3770 | 26.8 |
| 19 | 27.063 | 3.29215 | 91.7314 | 21.0 |
| 20 | 28.954 | 3.08127 | 58.2810 | 9.8 |
| 21 | 31.135 | 2.87029 | 39.1191 | 5.1 |
| 22 | 34.181 | 2.62111 | 45.7945 | 8.2 |

The present application provides crystal Form T of formula (I-1) compound, characterized in that the X-ray powder diffraction pattern of the crystal Form T has characteristic diffraction peaks at the following 2θ angles: 6.347±0.200°, 11.735±0.200°, and 18.221±0.200°.

In some embodiments of the application, the X-ray powder diffraction pattern of the above crystal Form T has characteristic diffraction peaks at the following 2θ angles: 6.347±0.200°, 11.012±0.200°, 11.735±0.200°, 18.221±0.200°, and 20.525±0.200°.

In some embodiments of the application, the X-ray powder diffraction pattern of the above crystal Form T has characteristic diffraction peaks at the following 2θ angles: 6.347±0.200°, 11.012±0.200°, 11.735±0.200°, 15.525±0.200°, 17.028±0.200°, 18.221±0.200°, 20.525±0.200°, and 25.872±0.200°.

In some embodiments of the application, the X-ray powder diffraction pattern of the above crystal Form T has characteristic diffraction peaks at the following 2θ angles: 6.347°, 9.315°, 10.351°, 11.012°, 11.735°, 12.780°, 14.017°, 15.525°, 16.475°, 17.028°, 18.221°, 19.285°, 20.213°, 20.525°, 22.738°, 23.383°, 23.924°, 25.039°, 25.872°, 26.855°, and 31.419°.

In some embodiments of the application, the XRPD pattern of the above crystal Form T is substantively shown in FIG. 24.

In some embodiments of the application, the analysis data of the XRPD pattern of the above crystal Form T is shown in Table 20:

TABLE 20
Analysis data of the XRPD pattern for crystal
Form T of formula (I-1) compound

| | 2θ | Interplanar | Intensity | Relative |
|---|---|---|---|---|
| No. | angle (°) | spacing(Å) | (count) | intensity (%) |
| 1 | 6.347 | 13.91355 | 302.5810 | 100.0 |
| 2 | 9.315 | 9.48700 | 82.8924 | 14.8 |
| 3 | 10.351 | 8.53947 | 119.5920 | 27.8 |
| 4 | 11.012 | 8.02830 | 166.7030 | 46.4 |
| 5 | 11.735 | 7.53500 | 216.2150 | 66.7 |
| 6 | 12.780 | 6.92121 | 101.4320 | 21.0 |
| 7 | 14.017 | 6.31292 | 84.8673 | 14.2 |
| 8 | 15.525 | 5.70310 | 144.4370 | 34.6 |
| 9 | 16.475 | 5.37629 | 94.8204 | 11.6 |
| 10 | 17.028 | 5.20290 | 178.9130 | 43.4 |
| 11 | 18.221 | 4.86492 | 199.2640 | 48.8 |
| 12 | 19.285 | 4.59875 | 112.0000 | 13.0 |
| 13 | 20.213 | 4.38977 | 195.9160 | 47.3 |
| 14 | 20.525 | 4.32360 | 197.6230 | 48.5 |
| 15 | 22.738 | 3.90762 | 101.9050 | 14.3 |
| 16 | 23.383 | 3.80123 | 107.5840 | 18.0 |
| 17 | 23.924 | 3.71650 | 113.4000 | 22.0 |
| 18 | 25.039 | 3.55352 | 83.8571 | 11.0 |
| 19 | 25.872 | 3.44100 | 138.9840 | 32.5 |
| 20 | 26.855 | 3.31722 | 130.8860 | 29.9 |
| 21 | 31.419 | 2.84491 | 68.5739 | 12.2 |

The present application provides crystal Form U of formula (I-1) compound, characterized in that the X-ray powder diffraction pattern of the crystal Form U has characteristic diffraction peaks at the following 2θ angles: 4.486±0.200°, 9.021±0.200°, and 27.215±0.200°.

In some embodiments of the application, the X-ray powder diffraction pattern of the above crystal Form U has characteristic diffraction peaks at the following 2θ angles: 4.486±0.200°, 9.021±0.200°, 14.894±0.200°, 23.876±0.200°, and 27.215±0.200°.

In some embodiments of the application, the X-ray powder diffraction pattern of the above crystal Form U has characteristic diffraction peaks at the following 2θ angles: 4.486±0.200°, 9.021±0.200°, 14.894±0.200°, 18.620±0.200°, 19.150±0.200°, 22.042±0.200°, 23.876±0.2000, and 27.215±0.200°.

In some embodiments of the application, the X-ray powder diffraction pattern of the above crystal Form U has characteristic diffraction peaks at the following 2θ angles: 4.486°, 9.021°, 10.970°, 13.600°, 14.894°, 15.853°, 18.076°, 18.620°, 19.150°, 22.042°, 23.876°, 26.295°, 27.215°, 28.304°, 30.674°, and 31.304°.

In some embodiments of the application, the XRPD pattern of the above crystal Form U is substantively shown in FIG. 25.

In some embodiments of the application, the analysis data of the XRPD pattern of the above crystal Form U is shown in Table 21:

TABLE 21
Analysis data of the XRPD pattern for crystal
Form U of formula (I-1) compound

| | 2θ | Interplanar | Intensity | Relative |
|---|---|---|---|---|
| No. | angle (°) | spacing(Å) | (count) | intensity (%) |
| 1 | 4.486 | 19.68043 | 275.2430 | 28.6 |
| 2 | 9.021 | 9.79491 | 637.5440 | 100.0 |
| 3 | 10.970 | 8.05867 | 83.1684 | 6.4 |
| 4 | 13.600 | 6.50555 | 152.6370 | 16.2 |
| 5 | 14.894 | 5.94312 | 237.0100 | 29.9 |
| 6 | 15.853 | 5.58570 | 94.9098 | 6.1 |
| 7 | 18.076 | 4.90347 | 153.0000 | 13.9 |
| 8 | 18.620 | 4.76163 | 200.6030 | 21.2 |
| 9 | 19.150 | 4.63092 | 169.3850 | 15.4 |
| 10 | 22.042 | 4.02941 | 170.6670 | 14.3 |
| 11 | 23.876 | 3.72385 | 230.6860 | 23.9 |
| 12 | 26.295 | 3.38653 | 150.7410 | 11.4 |
| 13 | 27.215 | 3.27417 | 245.4000 | 28.1 |
| 14 | 28.304 | 3.15058 | 132.0300 | 10.4 |
| 15 | 30.674 | 2.91232 | 149.8120 | 15.5 |
| 16 | 31.304 | 2.85515 | 108.2900 | 8.8 |

The present application provides crystal Form V of formula (I) compound, characterized in that the X-ray powder diffraction pattern of the crystal Form V has characteristic diffraction peaks at the following 2θ angles: 3.729±0.200°, 11.208±0.200°, and 23.480±0.200°.

In some embodiments of the application, the X-ray powder diffraction pattern of the above crystal Form V has characteristic diffraction peaks at the following 2θ angles: 3.729±0.200°, 11.208±0.200°, 15.943±0.200°, 21.259±0.200°, and 23.480±0.200°.

In some embodiments of the application, the X-ray powder diffraction pattern of the above crystal Form V has characteristic diffraction peaks at the following 2θ angles: 3.729±0.200°, 11.208±0.200°, 15.943±0.200°, 18.814±0.200°, 19.613±0.200°, 20.013±0.200°, 21.259±0.200°, and 23.480±0.200°.

In some embodiments of the application, the X-ray powder diffraction pattern of the above crystal Form V has characteristic diffraction peaks at the following 2θ angles: 3.729°, 7.556°, 7.930°, 11.208°, 14.132°, 14.587°, 15.943°, 17.855°, 18.814°, 19.613°, 20.013°, 21.259°, 21.476°, 21.966°, 23.271°, 23.480°, 24.340°, 25.142°, 25.409°, 26.267°, 26.849°, and 27.469°.

In some embodiments of the application, the XRPD pattern of the above crystal Form V is substantively shown in FIG. 26.

In some embodiments of the application, the analysis data of the XRPD pattern of the above crystal Form V is shown in Table 22:

TABLE 22
Analysis data of the XRPD pattern for crystal
Form V of formula (I) compound

| | 2θ | Interplanar | Intensity | Relative |
|---|---|---|---|---|
| No. | angle (°) | spacing(Å) | (count) | intensity (%) |
| 1 | 3.729 | 23.67427 | 242.1410 | 100.0 |
| 2 | 7.556 | 11.69024 | 83.8716 | 42.2 |
| 3 | 7.930 | 11.13948 | 88.0762 | 47.4 |
| 4 | 11.208 | 7.88802 | 127.0000 | 94.0 |
| 5 | 14.132 | 6.26178 | 67.8750 | 31.6 |
| 6 | 14.587 | 6.06759 | 71.8008 | 33.6 |
| 7 | 15.943 | 5.55451 | 128.7020 | 87.1 |
| 8 | 17.855 | 4.96390 | 83.5592 | 38.0 |
| 9 | 18.814 | 4.71291 | 117.4130 | 66.7 |
| 10 | 19.613 | 4.52252 | 119.2850 | 67.3 |
| 11 | 20.013 | 4.43305 | 109.0720 | 57.6 |
| 12 | 21.259 | 4.17610 | 134.0000 | 84.3 |
| 13 | 21.476 | 4.13434 | 106.0720 | 58.1 |
| 14 | 21.966 | 4.04311 | 84.5577 | 39.7 |
| 15 | 23.271 | 3.81932 | 125.2840 | 78.7 |
| 16 | 23.480 | 3.78580 | 134.8580 | 87.4 |
| 17 | 24.340 | 3.65402 | 101.7660 | 54.5 |
| 18 | 25.142 | 3.53917 | 65.6469 | 20.5 |
| 19 | 25.409 | 3.50260 | 72.1237 | 27.4 |
| 20 | 26.267 | 3.39006 | 60.2912 | 18.4 |
| 21 | 26.849 | 3.31788 | 78.8649 | 35.8 |
| 22 | 27.469 | 3.24437 | 71.5156 | 29.2 |

The present application further provides use of the above compounds or the crystal forms A, B, C, D, E, F, G, H, I, J, K, L, M, N, O, P, Q, R, S, T, U, V in the preparation of a medicament in treating diabetes, losing weight or NASH.

### Definitions and Explanations

Unless otherwise stated, the following terms and phrases used herein are intended to have the following meanings. A specific phrase or term should not be considered uncertain or unclear without a special definition, but should be understood in the ordinary sense. When a trade name appears herein, it refers to the corresponding commodity or its active ingredient.

The intermediate compounds of the application can be prepared by a variety of synthesis methods familiar to those skilled in the art, including the specific embodiments listed below, the embodiments formed by the combination of those specific embodiments with other chemical synthesis methods, and the equivalent alternative methods familiar to those skilled in the art. The preferred embodiments include but are not limited to the examples of the application.

The chemical reaction in the specific embodiment of the application is completed in a suitable solvent, and the solvent must be suitable for the chemical change in the application and the reagents and materials required by the application. In order to obtain the compound of the application, sometimes it is necessary for those skilled in the art to modify or select the synthesis step or reaction process on the basis of the existing embodiments.

The structure of the compound according to the application can be confirmed by conventional methods familiar to those skilled in the art. If the application relates to an absolute configuration of the compound, the absolute configuration can be confirmed by conventional technical means in the art. For example, single crystal X-ray diffraction (SXRD) is used to collect the diffraction intensity data of the cultured single crystal with the Bruker D8 venture diffractometer, wherein the light source is CuK α radiation, scanning mode: φ/ω scanning. After scanning and collecting relevant data, the absolute configuration can be confirmed by further analyzing the crystal structure with the direct method (Shelxs97).

The application will be described in detail below through examples, which do not mean any limitation to the application.

All solvents used in the application are commercially available, and can be used without further purification.

The solvent used in the application can be commercially available.

The application adopts the following abbreviations: aq stands for water; eq stands for equivalent; DCM stands for dichloromethane; PE stands for petroleum ether; DMF stands for N,N-dimethylformamide; DMSO stands for dimethyl sulfoxide; MEK stands for butanone; EtOH stands for ethanol; MeOH stands for methanol; IPA stands for isopropanol; MTBE stands for methyl tert-butyl ether; THF stands for tetrahydrofuran; Pd(dppf)Cl₂ stands for [1,1-bis(diphenylphosphino)ferrocene]palladium dichloride; Pd(OH)₂ stands for palladium hydroxide, LiAlH4 stands for lithium aluminum hydride; Tris stands for trometamol.

The compounds are named according to the general naming principles in this field or by using ChemDraw® software, and the commercially available compounds are named according to the supplier's catalog.

### Technical Effects

The compound of the application has stable crystal form, little influence by heat and humidity, good drug efficacy in vivo, and broad prospects for drug preparation; the compound of formula (I) shows a better ability to stimulate GLP-1 receptor.

### The X-Ray Powder Diffractometer (XRPD) Assay in the Application

1. Instrument model: X'Pert³ X-ray diffractometer of PANalytical
Test method: about 20 mg sample is used for XRPD detection.

The detailed parameters are as follows:

| Parameter | Set value |
|---|---|
| Instrument model | X'Pert³ |
| X-ray | Cu, kα, Kα1 (Å): 1.540598; Kα2 |
| | (Å): 1.544426; |
| X-ray tube setting | 45 kV, 40 mA |
| Divergence slit | Fixed ⅛° |
| Scanning Mode | Continuity |
| Scanning range (°2 Theta) | 3-40 |
| Scanning time per step (s) | 46.7 |
| Scanning step (°2 Theta) | 0.0263 |
| Test time (min) | 5 |

2. The X-ray diffraction pattern is acquired by D2 Phaser from Bruker Company, Germany. The parameters of the instrument are as follows.

| | 3 − 40 + 0.02 + | | Step |
|---|---|---|---|
| Method name | 0.2 + 15 · bsml | Induction mode | measurement |
| X-ray emitter | Cu, k-Alphal | Phototube | 30 |
| | (λ = 1.54184Å) | voltage (kV) | |
| Phototube | 10 | Divergence | 0.6 |
| current (mA) | | slit (mm) | |
| Axial Soller | 2.5 | Axial Soller | 2.5 |
| slit of main | | slit of secondary | |
| optical path (°) | | optical path (°) | |
| Detector slit (°) | 5.827 | Anti-scattering | 0 |
| | | slit (mm) | |
| Scanning axis | θs − θd | Step (deg) | 0.02 |
| Dwell time | 0.2 | Scanning | 3-40 |
| per step (S) | | range (deg) | |

### Differential Scanning Calorimeter (DSC) Assay in the Application

Instrument model: TA 2500 differential scanning calorimeter

Parameters and test methods are as follows:

| Parameter | Set value |
|---|---|
| Method | Linear temperature increase |
| Sample disk | Aluminum disk, Gland/non-gland |
| Temperature range | 25° C. to set end point temperature |
| Scanning rate (° C./min) | 10 |
| Protective gas | Nitrogen |

### Thermal Gravimetric Analyzer (TGA) Assay in the Application

Instrument model: TA 5500 thermogravimetric analyzer

The parameter and test method are as follows:

| Parameter | Set value |
|---|---|
| Method | Linear temperature increase |
| Sample disk | Aluminum disc, open |
| Temperature range | Room temperature to set |
| | end point temperature |
| Scanning rate (° C./min) | 10 |
| Protective gas | Nitrogen |

### Dynamic Vapor Sorption (DVS) Assay Used in the Application

The dynamic vapor sorption (DVS) curve is collected on the DVS Intrinsic plus by SMS (surface measurement systems). The relative humidity at 25° C. is corrected by the deliquescence point of lithium chloride (LiCl), magnesium nitrate [Mg(NO₃)₂] and potassium chloride (KCl).

The test parameters are as follows:

| Parameter | Set value | |
|---|---|---|
| Temperature | 25° | C. |
| Sample amount | 10-20 | mg |
| Protective gas and flow rate | N₂, 200 mL/min | |
| dm/dt | 0.002%/min | |
| Minimum dm/dt | 10 | min |

| Parameter | Set value |
|---|---|
| equilibration time | |
| Maximum | 180 min |
| equilibration time | |
| RH test range | 0% RH-95% RH |
| RH gradient | 10% (0% RH-90% RH, 90% RH-0% RH) |
| | 5% (90% RH-95% RH, 95% RH-90% RH) |

The hygroscopicity evaluation is classified as follows:

| Hygroscopicity classification | ΔW % |
|---|---|
| Deliquescence | Absorbing enough water |
| | to form liquid |
| Very hygroscopic | ΔW % ≥ 15% |
| Hygroscopic | 15% > ΔW % ≥ 2% |
| Slightly hygroscopic | 2% > ΔW % ≥ 0.2% |
| No or almost no hygroscopicity | ΔW % < 0.2% |

Note:
ΔW % stands for the hygroscopic weight gain of the test sample at 25 ± 1° C. [symbol] 80 ± 2% RH.

### BRIEF DESCRIPTION OF THE DRAWINGS

FIG. 1 is XRPD pattern of Cu-Kα radiation for crystal Form A of formula (11-1) compound;

FIG. 2 is DSC thermogram for crystal Form A of formula (II-1) compound;

FIG. 3 is TGA spectrum for crystal Form A of formula (II-1) compound;

FIG. 4 is XRPD pattern of Cu-Kα radiation for crystal Form B of formula (II) compound;

FIG. 5 is XRPD pattern of Cu-Kα radiation for crystal Form C of formula (II) compound;

FIG. 6 is XRPD pattern of Cu-Kα radiation for crystal Form D of formula (II) compound;

FIG. 7 is XRPD pattern of Cu-Kα radiation for crystal Form E of formula (II) compound;

FIG. 8 is DSC spectrum for crystal Form E of formula (II) compound;

FIG. 9 is TGA spectrum for crystal Form E of formula (II) compound;

FIG. 10 is XRPD pattern of Cu-Kα radiation for crystal Form F of formula (II) compound;

FIG. 11 is XRPD pattern of Cu-Kα radiation for crystal Form G of formula (II) compound;

FIG. 12 is XRPD pattern of Cu-Kα radiation for crystal Form H of formula (II) compound;

FIG. 13 is XRPD pattern of Cu-Kα radiation for crystal Form I of formula (II) compound;

FIG. 14 is XRPD pattern of Cu-Kα radiation for crystal Form J of formula (III) compound;

FIG. 15 is XRPD pattern of Cu-Kα radiation for crystal Form K of formula (IV-1) compound;

FIG. 16 is XRPD pattern of Cu-Kα radiation for crystal Form L of formula (IV) compound;

FIG. 17 is XRPD pattern of Cu-Kα radiation for crystal Form M of formula (V) compound;

FIG. 18 is XRPD pattern of Cu-Kα radiation for crystal Form N of formula (VI) compound;

FIG. 19 is XRPD pattern of Cu-Kα radiation for crystal Form O of formula (VII) compound;

FIG. 20 is XRPD pattern of Cu-Kα radiation for crystal Form P of formula (VII) compound;

FIG. 21 is XRPD pattern of Cu-Kα radiation for crystal Form Q of formula (VII) compound;

FIG. 22 is XRPD pattern of Cu-Kα radiation for crystal Form R of formula (I) compound;

FIG. 23 is XRPD pattern of Cu-Kα radiation for crystal Form S of formula (I) compound;

FIG. 24 is XRPD pattern of Cu-Kα radiation for crystal Form T of formula (I-1) compound;

FIG. 25 is XRPD pattern of Cu-Kα radiation for crystal Form U of formula (I-1) compound;

FIG. 26 is XRPD pattern of Cu-Kα radiation for crystal Form U of formula (I) compound;

FIG. 27 is DVS spectrum for crystal Form A of formula (II-1) compound;

FIG. 28 is DVS spectrum for crystal Form E of formula (II) compound;

FIG. 29 is DVS spectrum for crystal Form Q of formula (VIII) compound.

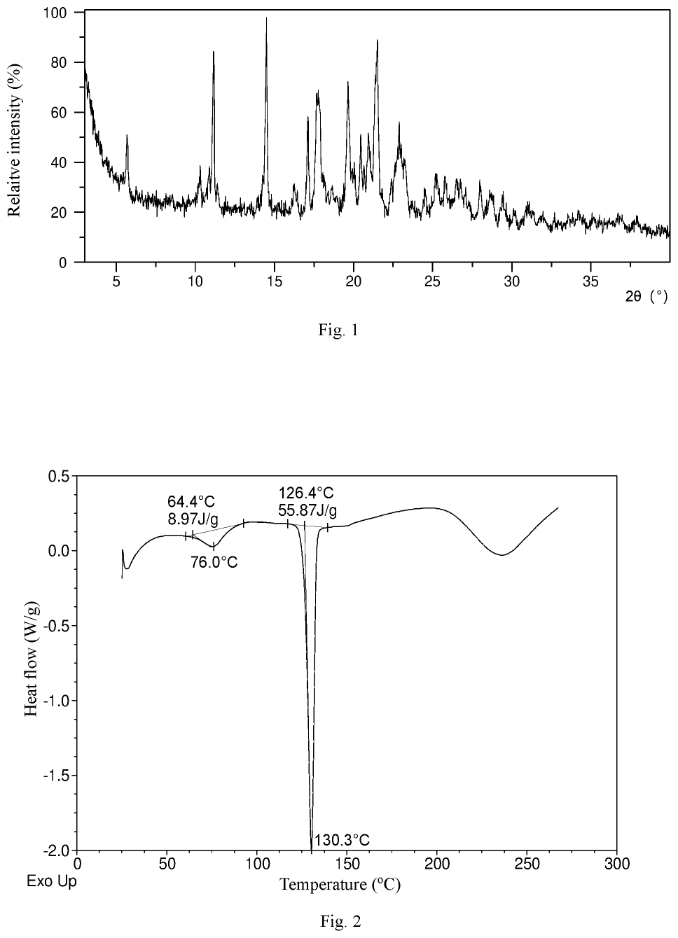

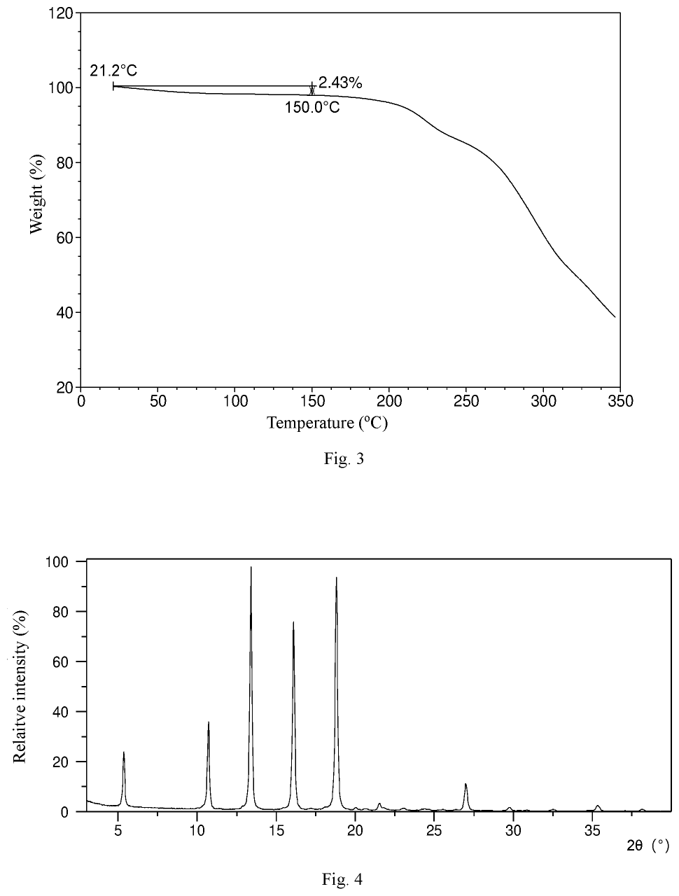

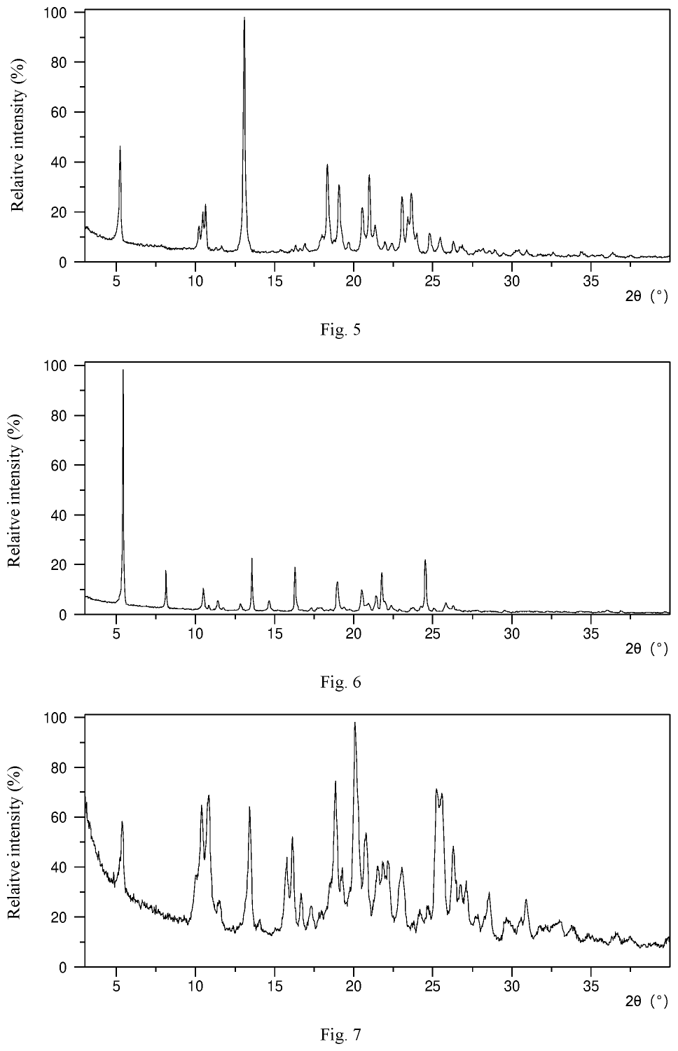

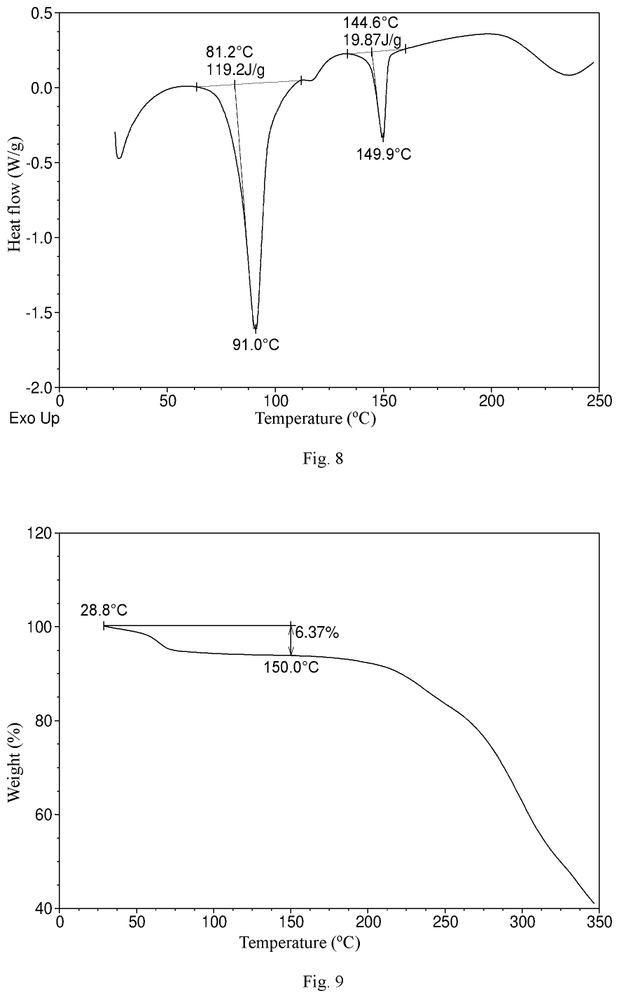

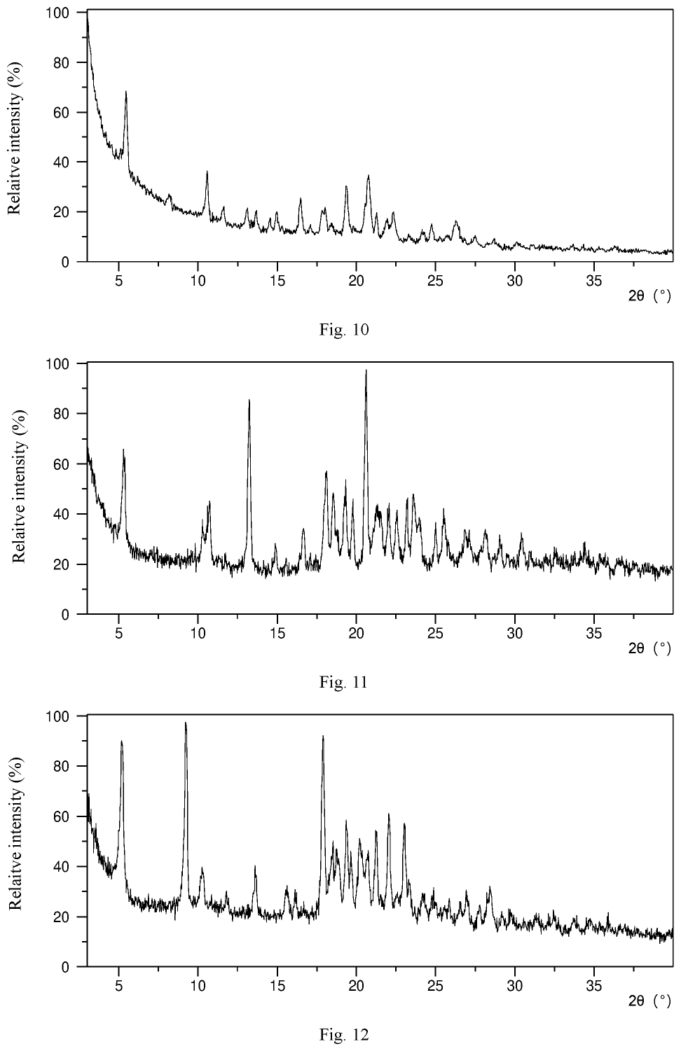

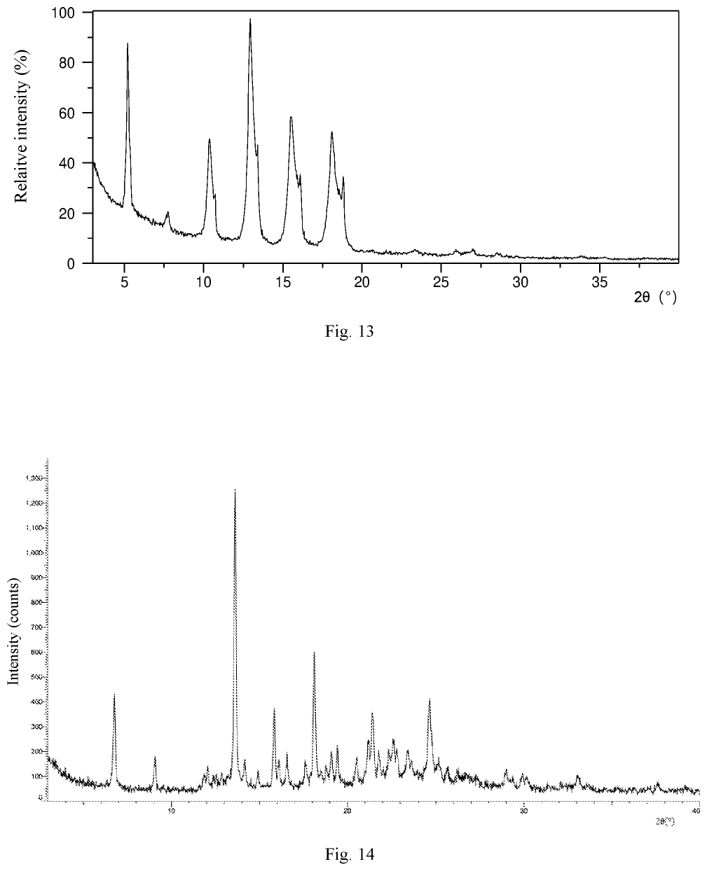

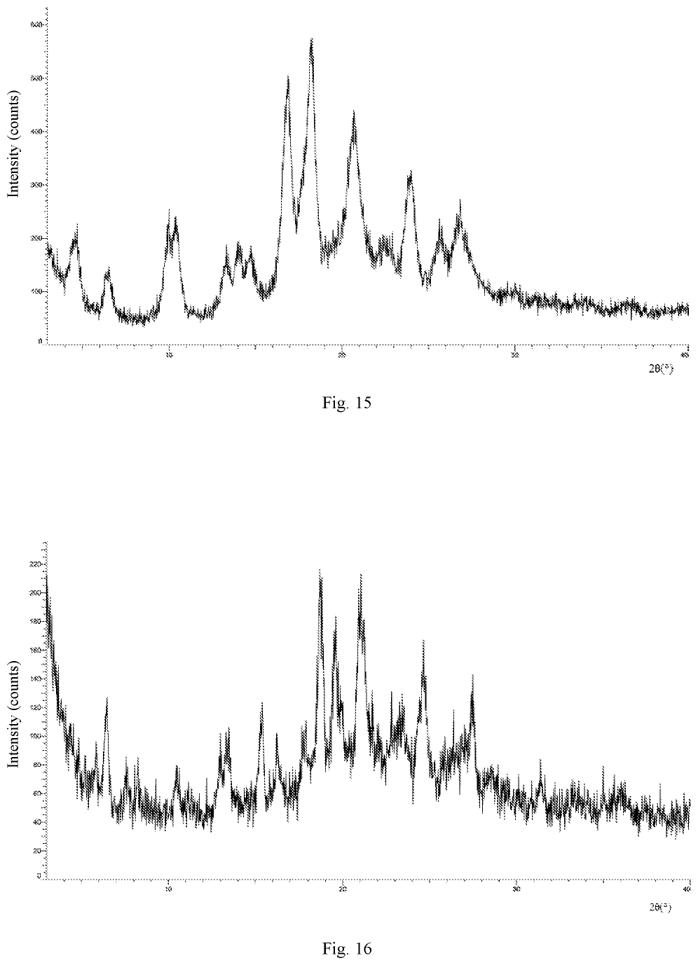

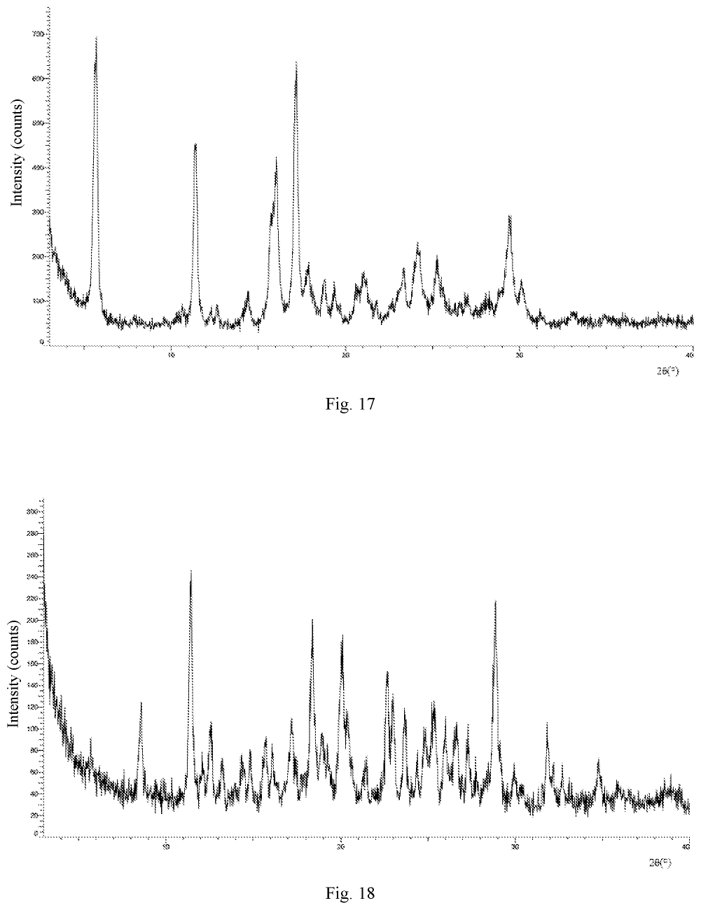

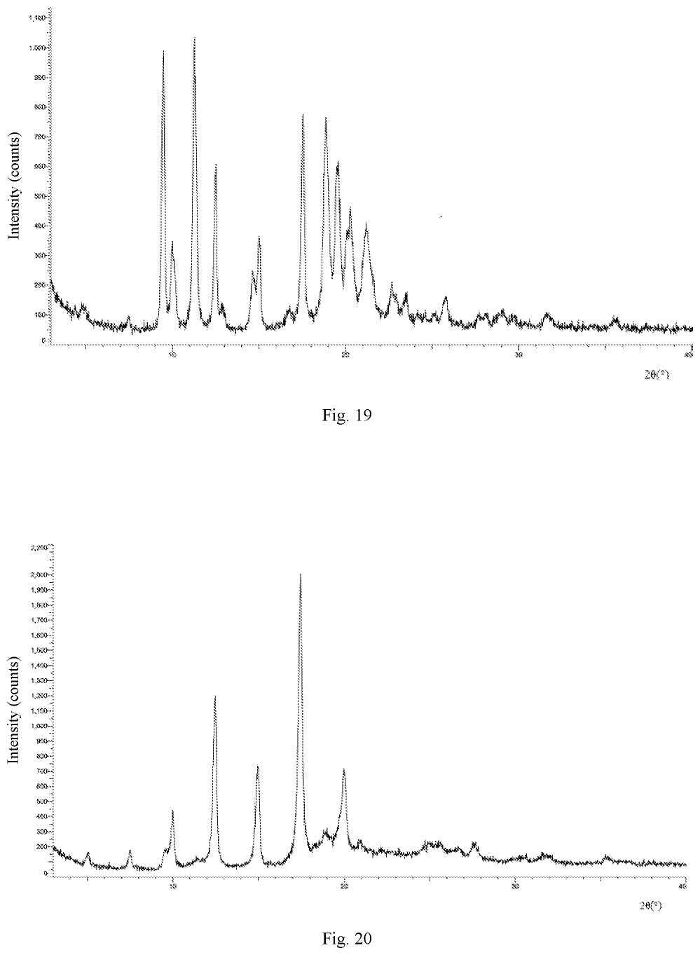

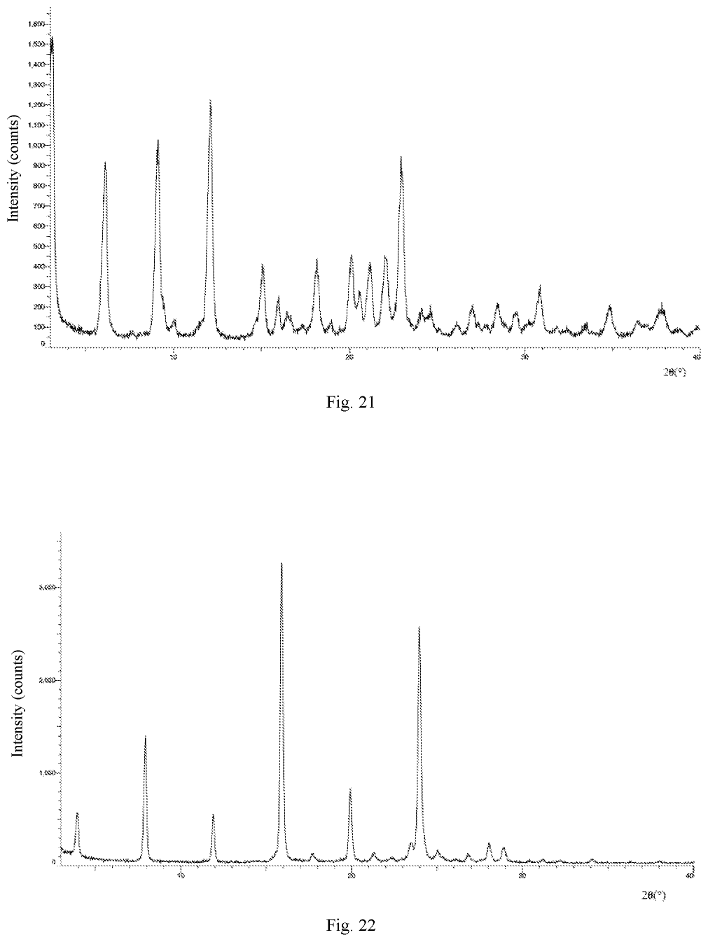

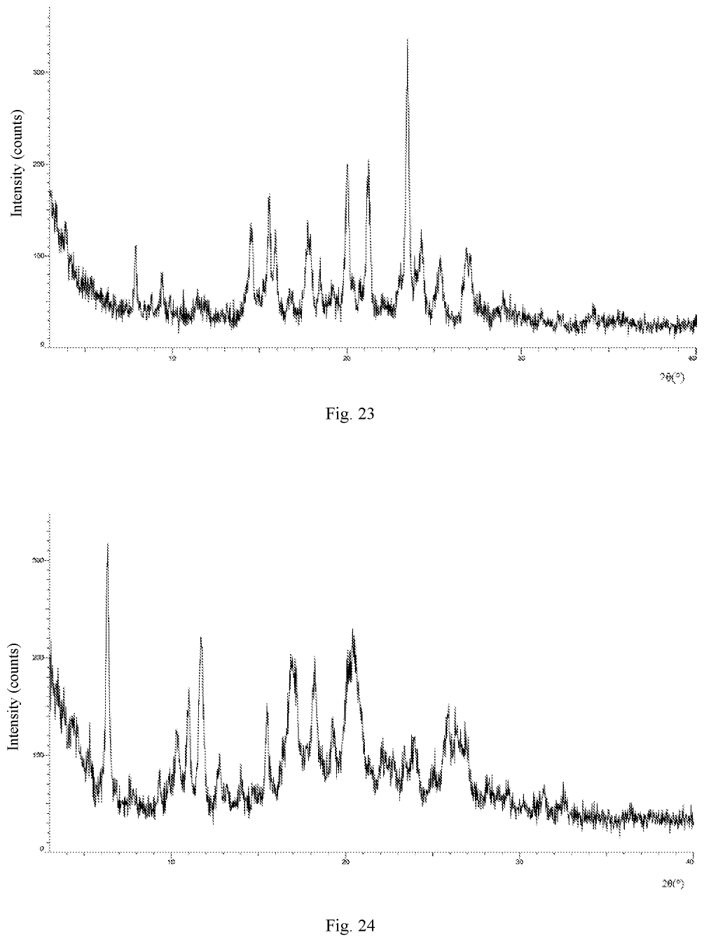

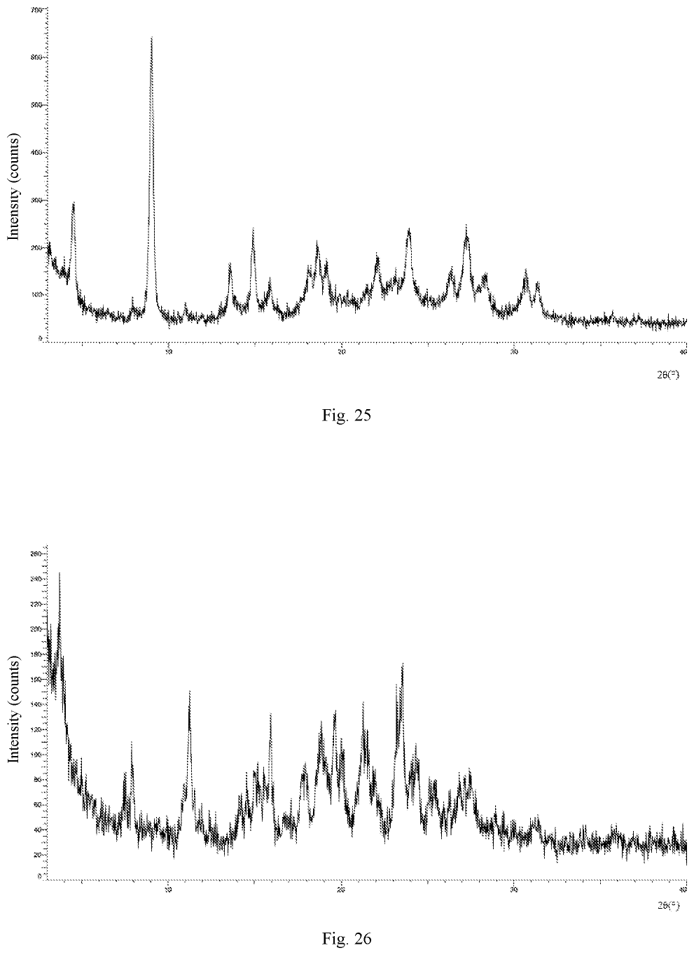

### SPECIFIC EMBODIMENTS

In order to better understand the content of the application, the application is further described in combination with specific examples which are not intended to limit the content of the application.

### Example 1: Synthesis of Formula (I) Compound

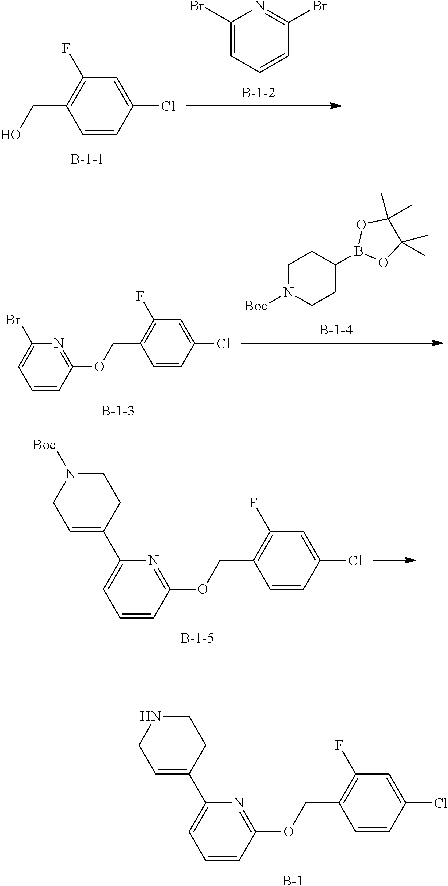

### Step 1: Synthesis of Compound B-1-3

B-1-1 (1.00 g, 6.23 mmol, 1 eq) was added into a reaction flask containing THF (40 mL), adding NaH (375 mg, 9.38 mmol, 60% content, 1.51 eq) under the protection of nitrogen at 0° C. to heat up to 22° C., stirring for 1 hour; adding B-1-2 (1.5 g, 6.33 mmol, 1.02 eq) to heat up to 60° C., stirring for 16 h. 20 mL of water was added to the reaction solution for quenching, adding DCM (20 mL*3) for extraction, collecting the organic phase to dry over anhydrous sodium sulfate, then concentrating to obtain a crude product. The crude product was separated and purified by column chromatography (petroleum ether: ethyl acetate=1:0) to obtain B-1-3. LCMS: m/z=317.8 [M+H]⁺.

### Step 2: Synthesis of Compound B-1-5

B-1-3 (1.50 g, 4.74 mmol, 1 eq), B-1-4 (1.50 g, 4.85 mmol, 1.02 eq), sodium carbonate (1.50 g, 14.15 mmol, 2.99 eq), dioxane (30 mL), and water (6 mL) were added into a reaction flask, adding Pd(dppf)Cl₂ (0.17 g, 232.33 μmol, 0.05 eq) under nitrogen atmosphere; the reaction system was stirred at 100° C. for 3 h, concentrating the reaction solution to obtain a crude product, adding water (50 mL) and extracting with ethyl acetate (50 mL*3); then combining the organic phases to wash with saturated sodium chloride aqueous solution (50 mL), drying over anhydrous sodium sulfate, filtering, and then concentrating the filtrate to obtain a crude product. The crude product was separated and purified by column chromatography (petroleum ether: ethyl acetate=1:0-20:1) to obtain B-1-5. LCMS: m/z=419.2 [M+H]⁺.

### Step 3: Synthesis of Compound B-1

B-1-5 (1.80 g, 4.30 mmol, 1 eq) and anhydrous DCM (30 mL) were added into a reaction flask, adding trifluoroacetic acid (7.70 g, 67.53 mmol, 5.0 mL, 15.72 eq); and the reaction system was stirred at 20° C. for 12 h. Sodium carbonate solution (30 mL) was added to the reaction solution, adding solid sodium carbonate to adjust pH of the solution to about 9-10, extracting with ethyl acetate (30 mL*3); then combining the organic phase to wash with saturated sodium chloride aqueous solution (30 mL), drying over anhydrous sodium sulfate, filtering, and then concentrating the filtrate to obtain a crude product. The crude product was separated and purified by column chromatography (DCM:MeOH=1:0-10:1) to obtain B-1. LCMS: m/z=319.1 [M+H]⁺.

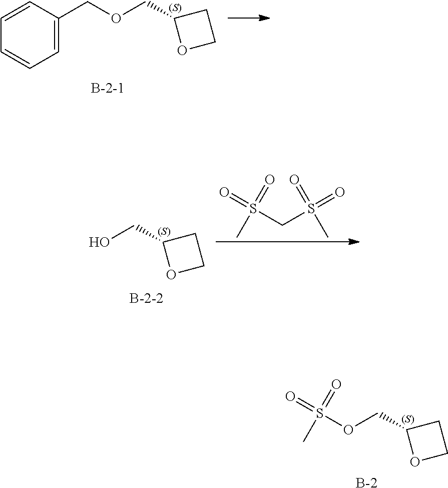

### Step 4: Synthesis of Compound B-2-2

Compound B-2-1 (12 g, 67.33 mmol, 1 eq) was dissolved in THF (120 mL), replacing argon, adding Pd(OH)₂ (6.00 g, 4.27 mmol, 10% content, 6.35e-2 eq), infusing hydrogen with press of 50 psi, and stirring at 45° C. for 24 h. The reaction solution was filtered with diatomite and washed with anhydrous THF to obtain a THF solution of B-2-2, carrying the next reaction directly without post-treatment. ¹H NMR (400 MHz, CDCl₃) δ ppm 7.10-7.16 (m, 2H), 7.03-7.07 (m, 2H), 4.75-4.83 (m, 1H), 4.52-4.61 (m, 1H), 4.39-4.46 (m, 1H).

### Step 5: Synthesis of Compound B-2

Compound B-2-2 (2 g, 22.70 mmol, 1 eq), and TEA (13.78 g, 136.20 mmol, 18.96 mL, 6 eq) were added into a reaction flask, after pumping and replacing nitrogen, adding methanesulfonic anhydride (11.86 g, 68.10 mmol, 2.64 mL, 3 eq) in batches at 0° C., then raising the temperature to 25° C. to react for 24 h. Water (125 mL) was poured into the reaction solution for quenching, separating the organic phase, extracting the aqueous phase with ethyl acetate (50 mL); then combining the organic phase to wash with saturated saline solution (100 mL), drying over anhydrous sodium sulfate, filtering, and then concentrating the filtrate with a water pump at 45° C. under reduced pressure to obtain a crude product. The crude product was separated and purified by column chromatography (PE:EA=1:0 to 1:1, gradient elution) to obtain B-2. ¹H NMR (400 MHz, CDCl₃) δ ppm 4.99-5.05 (m, 1H), 4.64-4.71 (m, 1H), 4.57 (dt, J=9.1, 6.08 Hz, 1H), 4.36 (d, J=3.88 Hz, 2H), 3.10 (s, 3H), 2.70-2.81 (m, 1H), 2.58-2.68 (m, 1H).

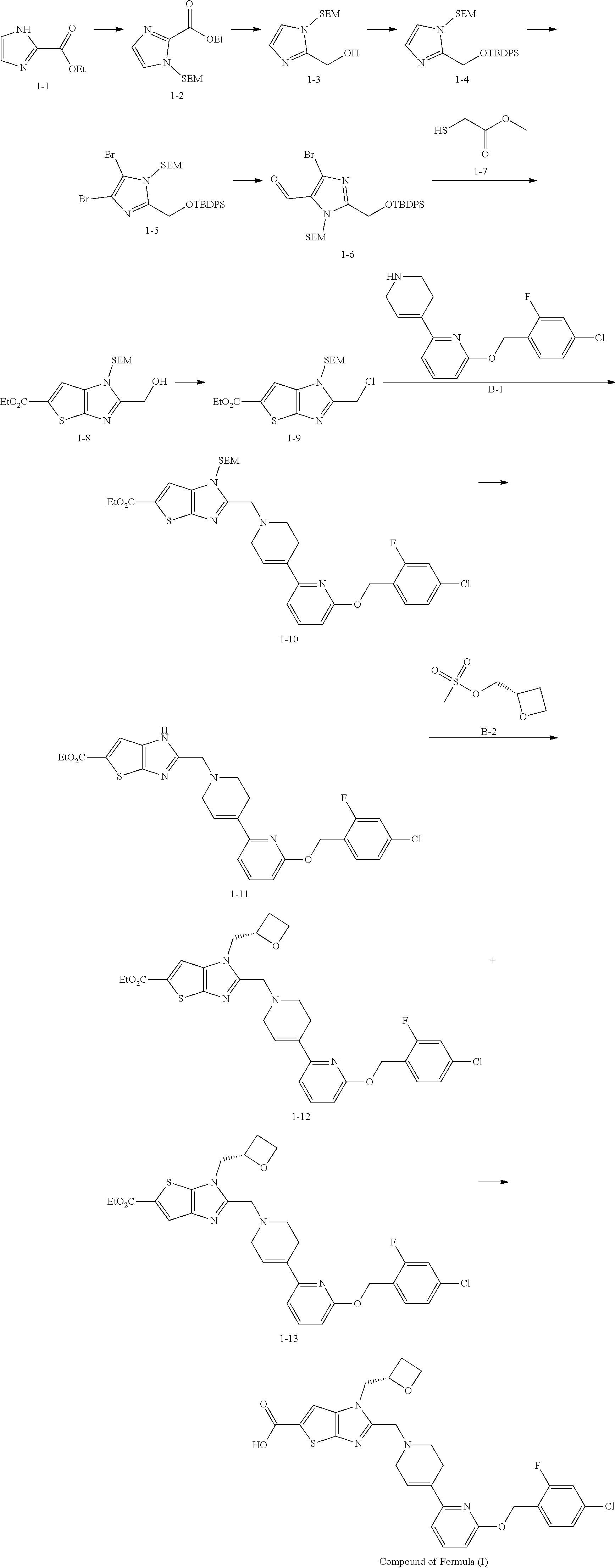

### Step 6: Synthesis of Compound 1-2

Compound 1-1 (23 g, 164.12 mmol, 1 eq) and DMF (115 mL) were added into a reaction flask, replacing nitrogen and cooling to 0° C., then adding NaH (9.85 g, 246.18 mmol, 60% content, 1.5 eq), replacing nitrogen again, adding 2-(trimethylsilyl)ethoxymethyl chloride (41.04 g, 246.18 mmol, 43.57 mL, 1.5 eq) dropwise, then raising the temperature to 25° C. to react for 12 h. The reaction solution was quenched with ice water (500 mL), extracting with ethyl acetate (200 mL*3); then combining the organic phases to wash with saturated saline solution (200 mL*2), drying over anhydrous sodium sulfate, filtering, and then concentrating the filtrate with a water pump at 45° C. under reduced pressure to obtain a crude product. The crude product was separated and purified by column chromatography (PE:EA=1:0 to 1:1, gradient elution) to obtain 1-2. ¹H NMR (400 MHz, CDCl₃) δ ppm 7.24 (d, J=0.63 Hz, 1H), 7.17 (s, 1H), 5.76 (s, 2H), 4.39 (q, J=7.13 Hz, 2H), 3.49-3.56 (m, 2H), 1.40 (t, J=7.13 Hz, 3H), 0.86-0.93 (m, 2H), −0.07-−0.04 (m, 9H).

### Step 7: Synthesis of Compound 1-3

THF (1000 mL) was added into the reaction flask, adding LiAlH₄ (6.04 g, 159.21 mmol, 1.5 eq) in batches, replacing nitrogen and cooling to 0° C., stirring for 15 min, then adding compound 1-2 (28.7 g, 106.14 mmol, 1 eq) at 0° C., and then heating up to 25° C. to react for 0.5 h. The reaction solution was cooled to 0° C., then 6 mL of water, 6 mL of 15% sodium hydroxide and 18 mL of water were successively added, heating to 25° C. and stirring for 15 min, then adding anhydrous magnesium sulfate to stir for 15 min, and then filtering to collect the filtrate, washing the filtrate with saturated saline solution (500 mL), drying over anhydrous sodium sulfate, filtering, and then concentrating the filtrate with a water pump at 45° C. under reduced pressure to obtain compound 1-3. ¹H NMR (400 MHz, CDCl₃) δ ppm 6.98 (d, J=1.13 Hz, 1H), 6.93 (d, J=1.13 Hz, 1H), 5.37 (s, 2H), 4.72 (s, 2H), 3.52 (dd, J=8.76, 7.75 Hz, 2H), 0.89-0.95 (m, 2H), −0.02-−0.01 (m, 9H).

### Step 8: Synthesis of Compound 1-4

Compound 1-3 (18.81 g, 82.37 mmol, 1 eq), tert-butyl diphenylchlorosilane (27.17 g, 98.84 mmol, 25.39 mL, 1.2 eq), imidazole (14.02 g, 205.92 mmol, 2.5 eq), and DMF (188 mL) were added into a reaction flask, replacing nitrogen to react at 25° C. for 16 h. Water (1000 mL) was poured into the reaction solution for quenching, adding ethyl acetate (200 mL*3) for extraction, combining the organic phases to wash with saturated saline solution (200 mL*3), drying over anhydrous sodium sulfate, filtering, and then concentrating the filtrate with a water pump at 45° C. under reduced pressure to obtain a crude product. The crude product was separated and purified by column chromatography (PE:EA=1:0 to 1:1, gradient elution) to obtain 1-4. ¹H NMR (400 MHz, CDCl₃) δ ppm 7.66-7.72 (m, 4H), 7.37-7.46 (m, 6H), 6.96-7.02 (m, 2H), 5.41 (s, 2H), 4.84 (s, 2H), 3.41-3.48 (m, 2H), 1.06 (s, 9H), 0.85-0.91 (m, 2H), −0.03 (s, 9H).

### Step 9: Synthesis of Compound 1-5

Compound 1-4 (22 g, 47.13 mmol, 1 eq) and THF (440 mL) were added into a reaction flask, after pumping and replacing nitrogen, adding N-bromosuccinimide (25.17 g, 141.40 mmol, 44.18 μL, 3 eq) in batches at 0° C., then raising the temperature to 25° C. to react for 12 h. The reaction solution was quenched with water (440 mL), extracting with ethyl acetate (2200 mL*2), then combining the organic phases to wash with saturated saline solution (2200 mL*1), drying over anhydrous sodium sulfate, filtering, and then concentrating the filtrate with a water pump at 30° C. under reduced pressure to obtain a crude product. The crude product was separated and purified by column chromatography (PE:EA=1:1 to 2:1, gradient elution) to obtain 1-5. ¹H NMR (400 MHz, CDCl₃) δ ppm 7.64-7.67 (m, 4H), 7.37-7.48 (m, 6H), 5.44 (s, 2H), 4.80 (s, 2H), 3.45-3.51 (m, 2H), 1.07 (s, 9H), 0.85-0.90 (m, 2H), −0.02 (s, 9H).

### Step 10: Synthesis of Compound 1-6

Compound 1-5 (6 g, 9.61 mmol, 1 eq) and THF (60 mL) were added into a reaction flask, replacing nitrogen and cooling down to −40° C., adding i-PrMgCl-LiCl (1.3 M, 8.13 mL, 1.1 eq) dropwise to stir for 1.5 h, then adding DMF (61.62 g, 843.09 mmol, 64.86 mL, 87.76 eq) dropwise, heating up to 25° C. and continuing to stir for 30 min. The reaction solution was quenched with water (120 mL), extracting with ethyl acetate (50 mL*2), combining the organic phases to wash with saturated saline solution (100 mL), drying over anhydrous sodium sulfate, filtering, and then concentrating the filtrate with a water pump at 45° C. under reduced pressure to obtain a crude product. The crude product was separated and purified by column chromatography (PE:EA=1:0 to 10:1, gradient elution) to obtain 1-6. ¹H NMR (400 MHz, CDCl₃) δ ppm 9.76 (s, 1H), 7.64-7.68 (m, 5H), 7.38-7.42 (m, 5H), 5.85 (s, 2H), 4.86 (s, 2H), 3.49-3.54 (m, 2H), 1.07 (s, 9H), 0.84-0.88 (m, 2H), −0.03 (s, 9H).

### Step 11: Synthesis of Compound 1-8

Compound 1-6 (1.23 g, 2.14 mmol, 1 eq) was dissolved in EtOH (61.5 mL), adding sodium ethoxide (2.19 g, 6.43 mmol, 20% content, 3 eq) and compound 1-7 (273.10 mg, 2.57 mmol, 233.42 μL, 1.2 eq) to stir at 20° C. for 2 h, and then heating up to 80° C. for 12 h. The reaction solution was quenched with water (50 mL), extracting twice with ethyl acetate (25 mL), combining the organic phases to wash with saturated saline solution (50 mL), filtering, and then concentrating the filtrate with a water pump at 45° C. under reduced pressure to obtain a crude product. The crude product was separated and purified by column chromatography (PE:EA=1:0 to 10:1, gradient elution) to obtain compound 1-8. ¹H NMR (400 MHz, CDCl₃) δ ppm 7.72 (s, 1H), 5.54 (s, 2H), 4.91 (s, 2H), 4.39 (q, J=7.13 Hz, 2H), 3.55-3.61 (m, 2H), 2.30-2.64 (m, 1H), 1.40 (t, J=7.13 Hz, 3H), 0.91-0.96 (m, 2H), −0.02 (s, 9H).

### Step 12: Synthesis of Compound 1-9

Compound 1-8 (120 mg, 336.59 mol, 1 eq), triethylamine (102.18 mg, 1.01 mmol, 140.55 μL, 3 eq) and DCM (2 mL) were added into a reaction flask, after pumping and replacing nitrogen, adding methylsulfonyl chloride (57.84 mg, 504.89 mol, 39.08 μL, 1.5 eq) in batches at 0° C., then raising the temperature to 25° C. to react for 12 h. The reaction solution was directly concentrated under reduced pressure to obtain a crude product. The crude product was separated and purified by thin-layer chromatography silica gel plate (DCM:MeOH=20:1) to obtain compound 1-9. ¹H NMR (400 MHz, CDCl₃) δ ppm 7.72 (s, 1H), 5.57 (s, 2H), 4.85 (s, 2H), 4.39 (q, J=7.13 Hz, 2H), 3.56-3.61 (m, 2H), 1.40 (t, J=7.13 Hz, 3H), 0.92-0.97 (m, 2H), −0.02 (s, 9H).

### Step 13: Synthesis of Compound 1-10

Compound 1-9 (0.16 g, 426.72 mol, 1 eq), compound B-1 (0.30 g, 941.11 mol, 2.21 eq), potassium carbonate (0.10 g, 723.56 mol, 1.70 eq) and acetonitrile (10 mL) were successively added into a reaction flask, and the reaction system was stirred at 60° C. for 10 h. The reaction solution was concentrated to obtain a crude product, adding water (10 mL), extracting with ethyl acetate (10 mL*3), then combining the organic phases to wash with saturated sodium chloride aqueous solution (10 mL), drying over anhydrous sodium sulfate, filtering, and then concentrating the filtrate to obtain a crude product. The crude product was separated and purified by column chromatography (petroleum ether: ethyl acetate=1:0 to 5:1) to obtain compound 1-10. LCMS: m/z=657.3 [M+H]⁺; ¹H NMR (400 MHz, CDCl₃) δ ppm 7.74 (s, 1H), 7.55 (t, J=7.91 Hz, 1H), 7.43 (t, J=8.03 Hz, 1H), 7.08-7.17 (m, 2H), 6.94 (d, J=7.28 Hz, 1H), 6.72 (br s, 1H), 6.66 (d, J=8.28 Hz, 1H), 5.66 (s, 2H), 5.43 (s, 2H), 4.38 (q, J=7.03 Hz, 2H), 3.95 (s, 2H), 3.52-3.57 (m, 2H), 3.29 (br s, 2H), 2.79-2.85 (m, 2H), 2.61 (br s, 2H), 1.40 (t, J=7.03 Hz, 3H), 0.89-0.94 (m, 2H), −0.10-−0.03 (m, 9H).

### Step 14: Synthesis of Compound 1-11

Compound 1-10 (0.30 g, 456.43 mol, 1 eq) and anhydrous DCM (5.0 mL) were successively added into a reaction flask, then adding trifluoroacetic acid (1.54 g, 13.51 mmol, 1.0 mL, 29.59 eq); and the reaction system was stirred at 40° C. for 5 h. Sodium carbonate solution (10 mL) was added to the reaction solution, adding solid sodium carbonate to adjust the solution pH to about 9-10, extracting with ethyl acetate (10 mL*3); then combining the organic phases to wash with saturated sodium chloride aqueous solution (10 mL), drying over anhydrous sodium sulfate, filtering, and then concentrating the filtrate to obtain a crude product. The crude product was separated and purified by column chromatography (petroleum ether: ethyl acetate=10:1-1:1) to obtain 1-11. LCMS: m/z=527.2 [M+H]⁺; ¹H NMR (400 MHz, CDCl₃) δ ppm 10.08 (br s, 1H), 7.68 (br s, 1H), 7.57 (t, J=7.78 Hz, 1H), 7.43 (t, J=8.16 Hz, 1H), 7.09-7.17 (m, 2H), 6.96 (d, J=7.53 Hz, 1H), 6.73 (br s, 1H), 6.69 (d, J=8.28 Hz, 1H), 5.44 (s, 2H), 4.37 (q, J=7.11 Hz, 2H), 3.96 (s, 2H), 3.35 (br d, J=3.01 Hz, 2H), 2.83-2.91 (m, 2H), 2.68 (br s, 2H), 1.39 (t, J=7.15 Hz, 3H).

### Step 15: Synthesis of Compounds 1-12 and 1-13

Compound 1-11 (0.20 g, 379.50 mol, 1 eq), B-2 (0.35 g, 2.11 mmol, 5.55 eq), potassium carbonate (0.40 g, 1.23 mmol, 3.23 eq) and acetonitrile (5 mL) were successively added into a reaction flask, and the reaction system was stirred at 80° C. for 10 h. The reaction solution was concentrated to obtain a crude product, adding water (10 mL), extracting with ethyl acetate (10 mL*3), then combining the organic phases to wash with saturated sodium chloride aqueous solution (10 mL), drying over anhydrous sodium sulfate, filtering, and then concentrating the filtrate to obtain a crude product. The crude product was separated and purified by column chromatography (petroleum ether: ethyl acetate=1:0 to 1:1) to obtain a mixture of compounds 1-12 and 1-13. LCMS (retention time: 3.508): m/z=597.1 [M+H]⁺; LCMS (retention time: 3.566): m/z=597.3 [M+H]⁺.

### Step 16: Synthesis of Formula (I) Compound

A mixture of compounds 1-12 and 1-13 (90 mg, 150.73 mol, 1 eq), water (0.4 mL), and acetonitrile (2 mL) were successively added into a reaction flask, adding 1,5,7-triazabicyclo (4.4.0)dec-5-ene (50 mg, 359.20 mol, 2.38 eq); and the reaction system was stirred at 20° C. for 10 h. The reaction solution was concentrated to obtain a crude product. The crude product was separated and purified by TLC (DCM:MeOH=10:1) to obtain a mixture, and the mixture was separated by supercritical fluid chromatography (column DAICEL CHIRALPAK IG (250 mm*30 mm, 10 m); mobile phase: [0.1% NH₃H₂O MeOH]; CO₂: 55%-55%, min) to obtain Formula (I) compound (retention time: 6.815 min).

Detection method (chromatographic column: Chiralpak IG-3 50ᵢÁ 4.6 mm I.D., 3 m; mobile phase: A: CO₂ B: methanol (0.05% diethylamine), isogradient elution: methanol (0.05% diethylamine) 40%, flow rate: 4 mL/min, column temperature: 35° C., back pressure: 1500 psi). LCMS: m/z=569.2 [M+H]⁺; ¹H NMR (400 MHz, DMSO-d₆) δ ppm 7.68 (t, J=7.91 Hz, 1H), 7.55 (t, J=8.16 Hz, 1H), 7.40-7.50 (m, 2H), 7.30 (dd, J=8.28, 1.76 Hz, 1H), 7.08 (d, J=7.53 Hz, 1H), 6.68-6.77 (m, 2H), 5.39 (s, 2H), 4.97-5.08 (m, 1H), 4.52-4.60 (m, 1H), 4.40-4.50 (m, 2H), 4.28-4.38 (m, 1H), 3.74-3.92 (m, 2H), 3.18 (br s, 2H), 2.68 (br d, J=5.52 Hz, 2H), 2.58-2.66 (m, 1H), 2.32-2.38 (m, 1H); ¹H NMR (400 MHz, CD₃OD) δ ppm 7.64 (s, 1H), 7.62 (t, J=7.91 Hz, 1H), 7.48 (t, J=8.03 Hz, 1H), 7.16-7.24 (m, 2H), 7.05 (d, J=7.53 Hz, 1H), 6.71 (br s, 1H), 6.68 (d, J=8.03 Hz, 1H), 5.42 (s, 2H), 5.19 (br d, J=4.27 Hz, 2H), 4.36-4.45 (m, 1H), 3.93-4.05 (m, 2H), 2.82-2.90 (m, 2H), 2.68-2.76 (m, 1H), 2.63 (br s, 2H), 2.48 (br d, J=8.78 Hz, 1H).

It is identified by two-dimensional nuclear magnetic resonance NOE that, Cs-H is related to C10-H, and the structure of the product is correct.

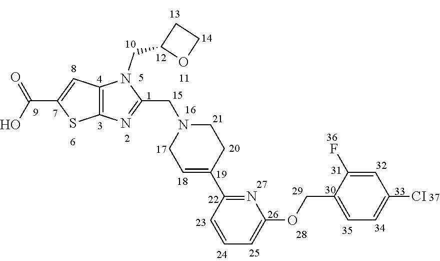

### Example 2: Preparation of Crystal Forms

### Preparation of Crystal Form A:

Trometamol (1.31 g, 10.8 mmol 1.02 eq) and methanol (30 mL) were successively added into a reaction flask, then stirring the system at 50° C. to dissolved clarification, cooling to 25° C. to add formula (I) compound (6 g, 10.54 mmol, 1 eq), and stirring the system to dissolved clarification; adding 90 mL of methyl tert-butyl ether to stir at 25° C. for 16 h, then filtering, and washing the filter cake with 12 mL mixed solvent of methanol: methyl tert-butyl ether=1:3 for three times, and then transferring the filter cake for vacuum drying at 50° C. for 16 h to obtain crystal Form A of formula (11-1) compound. ¹H NMR (400 MHz, CD₃OD) δ ppm 7.57-7.68 (m, 2H) 7.50 (t, J=7.91 Hz, 1H) 7.16-7.28 (m, 2H) 7.06 (d, J=7.53 Hz, 1H) 6.63-6.79 (m, 2H) 5.44 (s, 2H) 5.15-5.28 (m, 1H) 4.55-4.80 (m, 3H) 4.44 (dt, J=9.29, 6.02 Hz, 1H) 3.89-4.04 (m, 2H) 3.68 (s, 6H) 3.26 (br s, 2H) 2.68-2.86 (m, 3H) 2.63 (br s, 2H) 2.43-2.55 (m, 1H).

### Preparation of Crystal Form B:

About 15 mg of crystal Form A of formula (II-1) compound was weighed to add into an HPLC glass vial, then adding 0.5 mL H₂O; subjecting the obtained turbid solution to magnetic stirring (1000 rpm) at room temperature for about 4 days, then centrifuging to collect the solids and obtaining crystal Form B of formula (II) compound; after drying the sample open at room temperature overnight, it was transformed into crystal Form E.

### Preparation of Crystal Form C:

About 15 mg of formula (I) compound was weighed to add with equivalent molar ratio of Tris (3 mg) into an HPLC glass vial, then adding 0.5 mL EtOH; subjecting the obtained turbid solution to magnetic stirring (1000 rpm) at room temperature for about 4 days, then centrifuging to collect the solids and obtaining crystal Form C of formula (II) compound; after drying the sample open at room temperature overnight, it was transformed into crystal Form E.

### Preparation of Crystal Form D:

About 15 mg of crystal Form A of formula (II-1) compound was weighed to add into a 3 mL vial, then adding 1.0 mL MeOH/MTBE (1:1, v/v), stirring at 50° C. for equilibrating about 1 hour, and then filtering to obtain the supernatant. The obtained supernatant was placed in the biochemical incubator, then cooling from 50° C. to 5° C. at 0.1° C./min, and keeping the constant temperature at 5° C.; collecting the precipitated solids to obtain the crystal form D of formula (II) compound; after drying the sample open at room temperature overnight, it was transformed into crystal Form E.

### Preparation of Crystal Form E:

About 15 mg of crystal Form A of formula (II-1) compound was weighed to add into an HPLC glass vial, then adding 0.5 mL IPA, and subjecting the obtained suspension to magnetic stirring (1000 rpm) under the condition of temperature cycling (40° C.-5° C., 0.1° C./min, 2 cycles); and then centrifuging to collect the solids and obtaining crystal form E of formula (II) compound. ¹H NMR (400 MHz, CD₃OD) δ ppm 7.59-7.64 (m, 2H) 7.50 (t, J=8.03 Hz, 1H) 7.21 (t, J=10.16 Hz, 2H) 7.06 (d, J=7.28 Hz, 1H) 6.72-6.75 (m, 1H) 6.68 (d, J=8.29 Hz, 1H) 5.44 (s, 2H) 5.21 (qd, J=7.03, 3.01 Hz, 1H) 4.55-4.73 (m, 3H) 4.4 4 (dt, J=9.03, 6.02 Hz, 1H) 3.91-4.01 (m, 2H) 3.68 (s, 6H) 3.26 (br s, 2H) 2.70-2.84 (m, 3H) 2.63 (br s, 2H) 2.40-2.54 (m, 1H).

### Preparation of Crystal Form F:

About 15 mg of crystal Form A of formula (II-1) compound was weighed to add into a 20 mL vial, then dissolving the solids completely with 0.2-1.0 mL MeOH (filtering out the undissolved sample by 0.45 m PTFE filter to obtain clear solution). MTBE was added dropwise to the obtained clear solution while stirring (1000 rpm) until solids were precipitated to obtain crystal Form F of formula (II) compound; after drying the sample open at room temperature overnight, it was transformed into crystal Form E.

### Preparation of Crystal Form G:

About 15 mg of crystal Form A of formula (II-1) compound was weighed to add into a 3 mL vial, and about 3 mL of EtOH was added into another 20 mL vial, then placing the 3 mL vial open in the 20 mL vial, and then sealing the 20 mL vial; after standing at room temperature for 7 days, collecting the solids to obtain the crystal Form G of formula (II) compound; after drying the sample open at room temperature overnight, it was transformed into crystal Form E.

### Preparation of Crystal Form H:

About 15 mg of crystal Form A of formula (II-1) compound was weighed to add into a 3 mL vial, and about 3 mL of THF was added into another 20 mL vial, then placing the 3 mL vial open in the 20 mL vial, and then sealing the 20 mL vial; after standing at room temperature for 7 days, collecting the solids to obtain the crystal Form H of formula (II) compound; after drying the sample open at room temperature overnight, it was transformed into crystal Form E.

### Preparation of Crystal Form I:

About 15 mg of crystal Form A of formula (II-1) compound was weighed to add into an HPLC glass vial, then adding 0.5 mL H₂O, and subjecting the obtained suspension to magnetic stirring (1000 rpm) under the condition of temperature cycling (40° C.-5° C., 0.1° C./min, 2 cycles); and then centrifuging to collect the solids and obtain crystal form I of formula (II) compound; after drying the sample open at room temperature overnight, it was transformed into crystal Form E.

### Preparation of Crystal Form J:

About 50 mg of formula (I) compound was weighed to dissolve in 0.8 mL of butanone, then adding 0.9 mL of 0.1 mol/L phosphor-MEK solution at 50° C.; the solid was firstly dissolved to clarification and then precipitated at 50° C., stirring at 50° C. for 1 h and then slowly cooling to 18° C. (about 0.5 K/min); after stirring at 18° C. for about 18 h, filtering to obtain the solids, and drying at 50° C. for 3-4 h to obtain crystal Form J of formula (III) compound.

### Preparation of Crystal Form K:

About 50 mg of formula (I) compound was weighed to dissolve in 0.4 mL of butanone, then adding 0.9 mL of 0.1 mol/L methanol solution of oxalic acid at 50° C., and dissolving to clarification at 50° C., then cooling to 18° C. without precipitation; adding 3.0 mL n-heptane and layering; after stirring at 18° C. for about 18 h, the solids were precipitated, filtering to obtain the solids, and drying at 50° C. for 3-4 h to obtain crystal Form K of formula (IV) compound.

### Preparation of Crystal Form L:

About 50 mg of formula (I) compound was weighed to dissolve in 1.0 mL of ethyl acetate, then adding 8.43 mg of solid oxalic acid at 50° C.; the white solids were precipitated at 50° C., then stirring at 50° C. for 1 h to form an oil; slowly cooling to 18° C.; stirring at 18° C. for about 18 h to change into solids, then filtering to obtain the solids, and drying at 50° C. for 3-4 h to obtain crystal Form L of formula (IV) compound.

### Preparation of Crystal Form M:

About 50 mg of formula (I) compound was weighed to dissolve in 1.2 mL of butanone, then adding 10.18 mg of maleic acid at 18° C. to precipitate white solids, and continuing to stir for about 18 h; filtering to obtain the solids, and drying at 50° C. for 3-4 h to obtain crystal form M of formula (V) compound.

### Preparation of Crystal Form N:

About 50 mg of formula (I) compound was weighed to dissolve in 0.4 mL of butanone, then adding 0.9 mL of p-toluenesulfonic acid solution (0.1 mol/L methanol solution) at 18° C., and continuing to stir for about 18 h. Without precipitation, after adding 1.0 mL of n-heptane, the solids were precipitated, filtering to obtain the solids, and drying for 3-4 h at 50° C. to obtain crystal Form N of formula (VI) compound.

### Preparation of Crystal Form O:

About 50 mg of formula (I) compound was weighed to dissolve in 0.8 mL of butanone, then adding 0.9 mL of L-arginine solution (0.1 mol/L methanol solution) at 18° C., firstly dissolving to clarification, after white solids were rapidly precipitated, continuing to stir for about 18h; filtering to obtain the solids, and drying at 50° C. for 3-4 h to obtain crystal Form O of formula (VII) compound. ¹H NMR (400 MHz, CD3OD) δ ppm 7.60 (t, J=7.78 Hz, 1H) 7.57 (s, 1H) 7.48 (t, J=8.11 Hz, 1H) 7.15-7.23 (m, 2H) 7.04 (d, J=7.45 Hz, 1H) 6.71 (br s, 1H) 6.66 (d, J=8.33 Hz, 1H) 5.42 (s, 2H) 5.13-5.24 (m, 1H) 4.51-4.71 (m, 3H) 4.41 (dt, J=9.15, 5.95 Hz, 1H) 3.87-4.06 (m, 3H) 3.82 (dd, J=4.82, 1.75 Hz, 1H) 3.73-3.80 (m, 1H) 3.60-3.71 (m, 3H) 3.24 (br s, 2H) 3.15 (d, J=6.14 Hz, 2H) 2.76-2.84 (m, 2H) 2.66-2.75 (m, 4H) 2.61 (br s, 2H) 2.40-2.52 (m, 1H).

### Preparation of Crystal Form P:

About 50 mg of formula (I) compound was weighed to dissolve in 1.4 mL of acetonitrile, then adding 0.9 mL of L-arginine solution (0.1 mol/L methanol solution) at 18° C., firstly dissolving to clarification, after white solids were rapidly precipitated, continuing to stir for about 18h; filtering to obtain the solids, and drying at 50° C. for 3-4 h to obtain crystal Form P of formula (VII) compound.

### Preparation of Crystal Form Q:

About 50 mg of formula (I) compound was weighed to dissolve in 1.0 mL of acetonitrile, then adding 0.9 mL of meglumine solution (0.1 mol/L methanol solution) at 18° C., firstly dissolving to clarification, then adding 2.0 mL of methyl tert-butyl ether; after stirring for about 10 min, the solids were precipitated, then continuing to stir for about 18h; filtering to obtain the solids, and drying at 50° C. for 3-4 h to obtain crystal Form Q of formula (VIII) compound.

### Preparation of Crystal Form R:

About 50 mg of formula (I) compound was weighed to dissolve in 0.6 mL of tetrahydrofuran, after slowly adding 0.8 mL of acetonitrile to the solution at 17° C., the solids were precipitated, then filtering to obtain crystal Form R of formula (I) compound.

### Preparation of Crystal Form S:

About 50 mg of formula (I) compound was weighed to dissolve in 0.2 mL of tetrahydrofuran at 50° C., after stirring at 50° C. for a period of time, cooling to 20° C. at a certain cooling rate; solids were precipitated, then filtering to obtain the crystal Form S of formula (I) compound.

### Preparation of Crystal Form T:

About 50 mg of formula (I) compound was weighed, then adding 0.8 mL of methanol, keeping the system suspended and stirred at 20° C., and filtering the solid after a period of time to obtain crystal Form T of formula (I-1) compound.

### Preparation of Crystal Form U:

About 50 mg of formula (I) compound was weighed, then adding 0.8 mL of ethyl acetate, keeping the system suspended and stirred at 20° C., and filtering the solid after a period of time to obtain crystal Form U of formula (I-1) compound.

### Preparation of Crystal Form V:

About 50 mg of formula (I) compound was weighed, then adding 0.6 mL of acetonitrile, keeping the system suspended and stirred at 50° C., and filtering the solid after a period of time to obtain crystal Form V of formula (I) compound.

### Example 3: Study on the Hygroscopicity of the Crystal Forms

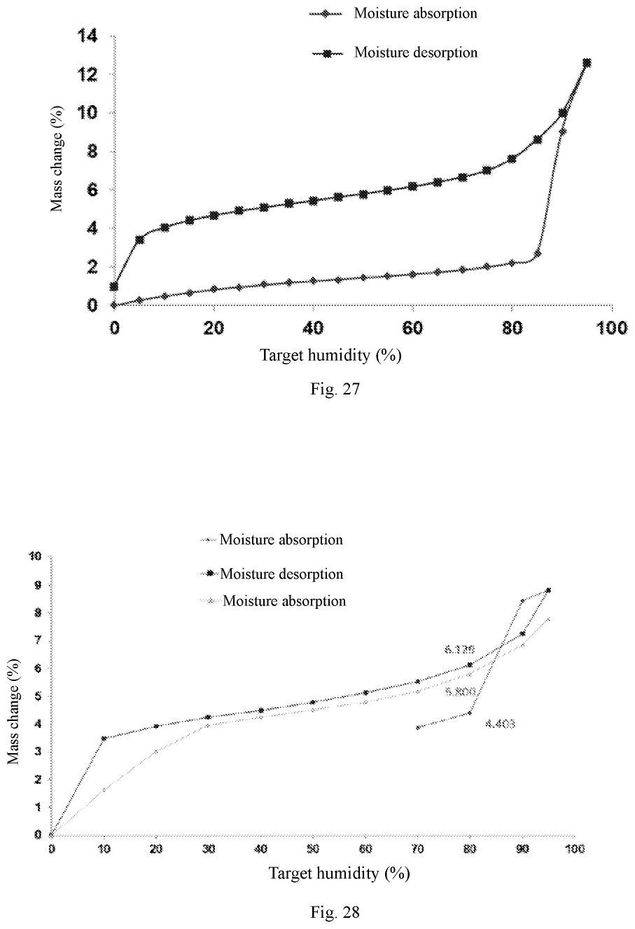

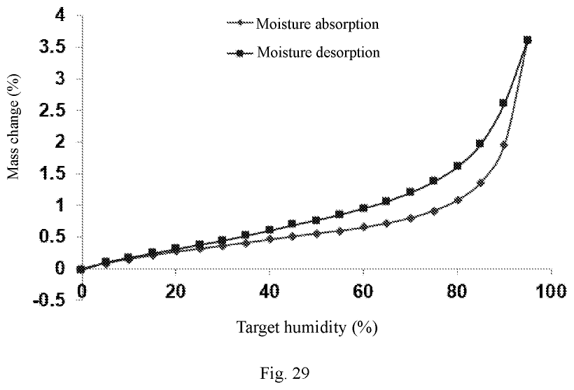

SMS Intrinsic dynamic vapor sorption instrument was used to respectively take 10 mg crystal Form A of formula (II-1) compound, crystal Form E of formula (II) compound and crystal Form Q of formula (VIII) compound to put into DVS sample disk for testing. The specific results are shown as the DVS spectrums in FIGS. 27-29.

It can be seen from the above that, the hygroscopic weight gain of crystal Form A of formula (II-1) compound at 25° C. and 80% RH is 2.20%, and crystal Form A is hygroscopic; the crystal form changes before and after adsorption and desorption. The hygroscopic weight gain of crystal form E of formula (II) compound is 2.20% at 25° C. and 80% RH, and crystal Form E is hygroscopic; the crystal form changes before and after adsorption and desorption. The hygroscopic weight gain of crystal form Q of formula (VIII) compound is 1.08% at 25° C. and 80% RH, and crystal Form Q is slightly hygroscopic; the crystal form remains unchanged before and after adsorption and desorption.

### Example 4: Study on the Solid Stability of Crystal Form E of Formula (II) Compound

500 mg crystal Form E of formula (II) compound was weighed to place at the bottom of a glass sample flask, then spreading into a thin layer. The crystal Form E was fully exposed when placing it under accelerated conditions (40TC/75% RH, and 60C/75% RH). Samples were taken from the above placed crystal Form E on day 5, day 15, month 1 and month 2 for XRPD detection, and the detection results were compared with the initial test results on day 0. The experimental results are shown in Table 23 below.

TABLE 23
Results of the study on solid stability of crystal Form E

| Experimental conditions | Time point | Crystal form |
|---|---|---|
| 60° C., 75% RH, open | Day 5 | crystal Form E |
| | Day 15 | crystal Form E |
| | Month 1 | crystal Form E |
| | Month 2 | crystal Form E |
| 40° C., 75% RH, open | Day 5 | crystal Form E |
| | Day 15 | crystal Form E |
| | Month 1 | crystal Form E |
| | Month 2 | crystal Form E |

Conclusion: crystal Form E of formula (II) compound has good stability.

### Example 5: Study on the Solid Stability of Crystal Form Q of Formula (VIII) Compound

500 mg crystal Form Q of formula (VIII) compound was weighed to place at the bottom of a glass sample flask, then spreading into a thin layer. The crystal Form Q was fully exposed when placing it under accelerated conditions (40° C./75% RH, and 60° C./75% RH). Samples were taken from the above placed crystal Form E on day 5, day 15 and month 1 for XRPD detection, and the detection results were compared with the initial test results on day 0. The experimental results are shown in Table 24 below.

TABLE 24
Results of the study on solid stability of crystal Form Q

| Experimental conditions | Time point | Crystal form |
|---|---|---|
| 60° C., 75% RH, open | Day 5 | crystal Form Q |
| | Day 15 | crystal Form Q |
| | Month 1 | crystal Form Q |
| 40° C., 75% RH, open | Day 5 | crystal Form Q |
| | Day 15 | crystal Form Q |
| | Month 1 | crystal Form Q |

Conclusion: crystal Form Q of formula (VIII) compound has good stability.

### Experimental Example 1: Detection of Cell Viability In Vitro

### 1. Materials

### 1) Cell Strain

The cell was constructed by Shanghai Wuxi AppTec New Drug Development Co., Ltd. Details are shown in the following table.

| Target | Host cell |
|---|---|
| GLP-1 | HEK293 |

### 2) Reagents

cAMP Detection Kit, Cisbio (Cat # 62AM4PEJ)
1M HEPES, Invitrogen (Cat # 15630-106)
1X HBSS, Invitrogen (Cat # 14025)
BSA, Sigma (Cat # B2064)
IBMX, Sigma (Cat # 15879)
Exenatide, Hao Yuan (HY-13443A)

### 3) Instruments

OptiPlate-384, White, PerkinElmer (Cat #6007290); 384 well plate for Echo, Labcyte (Cat #P-05525); EnVision, PerkinElmer; Vi-cell counter, Beckman (Cat # Vi-CELL™ XR Cell Viability Analyzer)

### 4) Compound Information

The compound was prepared to a working concentration of 30 M with DMSO. In this test, the use amount of each sample is 5 μL.

### 2. Methods

### 1) Experimental Materials

### Experimental Buffer

| | Final | |
|---|---|---|
| Volume | concentration | |
| 24.5 mL Hanks Buffer Saline Solution (HBSS) | 1x | |
| 125 μL HEPES 1M | 5 | mM |
| 333 μL 7.5% BSA Solution | 0.1% | |
| 25 μL IBMX 500 mmol/L | 0.5 | mmol/L |

Adjusting to pH 7.4, and diluting with HBSS 1x to 25 mL.

### Preparation of Detection Reagent

Preparation of cAMP detection reagent: 250 μL cAMP-D2, and 250 μL anti-cAMP cryptate reagent were added to 4 mL lysis buffer, then mixing well gently.

### 2) Experimental Methods

### a) Preparation of Compound Plate:

The compound to be tested was diluted 3 times at 10 points, the initial concentration was 30 M; and the dilution was completed by Bravo plateform.

The reference compound exenatide was diluted 3 times at 10 points, the initial concentration was 500 nM, and Bravo completed the dilution.

### b) Transferring the Compound:

1) 100 nL of the compound was transferred to OptiPlate-384 plate by Echo technology. 2) The OptiPlate-384 plate was centrifuged at 1000 rpm for 5 seconds.

### c) Preparation of Cell Suspension

1) A GLP-1 cell cryopreservation tube was quickly thawed in warm water at 37° C. 2) The cell suspension was transferred to a 15 mL Transfer centrifuge tube, then gently rinsing with 10 mL HBSS.
3) The centrifuge tube was centrifuged at room temperature and 1000 rpm for 1 min.
4) The supernatant was discarded.
5) The cells at the bottom were gently dispersed and washed with 10 mL HBSS, then centrifuging to obtain cell sediment, and resuspending the cells with experimental buffer.
6) Cell density and viability were measured by Vi-cell counter.
7) GLP-1 cells were diluted with experimental buffer to a concentration of 2.0*10⁵/mL.
8) 100 nL of diluted cell suspension was transferred into OptiPlate-384 plate.
9) The cells were incubated at room temperature for 30 min.

### d) Addition of Detection Reagent:

1) 10 μL of 800 nM gradient diluted cAMP standard was added to an empty well of OptiPlate-384 plate.
2) 10 μL cAMP detection reagent was added.
3) OptiPlate-384 plate was covered with TopSeal-A film, and incubated at room temperature for 60 min.

The TopSeal-A was removed for reading the plate in EnVision microplate reader.

The experimental results are shown in Table 25 below.

TABLE 25
Detection results of in vitro cell viability

| Compound | Human-GLP1, EC₅₀ (nM) |
|---|---|
| Formula (O) compound | 0.37 |

Conclusion: The compound of the application has better agitation ability for GLP-1 receptor

### Experimental Example 2: Study on DMPK in Rats

### Experimental Purpose:

Male SD rats were used as test animals. After single administration, the plasma concentration of the compound was measured, and the pharmacokinetic behavior was evaluated.

### Experimental Operations:

Two healthy adult male SD rats were selected as the oral group. In the oral group, the vehicle was 20% PEG400/10% solutol/70% water; after the compound to be tested was mixed with the vehicle, 0.5 mg/mL clear solution was prepared by vortex and ultrasound. After oral administration at 5 mg/kg, the whole blood of rat was collected for a certain period of time to prepare plasma; then the drug concentration was analyzed by LC-MS/MS method, and the pharmacokinetic parameters were calculated by Phoenix WinNonlin software (Pharsight, USA). The experimental results are shown in Table 26 below.

TABLE 26
PK detection results of the compound of the application

| | Cₘₐₓ | AUC₀₋ₗₐₛₜ | |
|---|---|---|---|
| Compound No. | (nM) | (h*nmol/L) | F % |
| Formula (O) compound | 85.5 | 234 | 6.6% |
| Crystal Form A of Formula | 174 | 476 | 11.5% |
| (II-1) compound | | | |
| Crystal Form Q of Formula | 185 | 530 | 13.6% |
| (VIII) compound | | | |

Note:
PEG stands for polyethylene glycol; solutol stands for polyethylene glycol-15 hydroxystearate; Cₘₐₓ is the maximum concentration; AUC₀₋ₗₐₛₜ is the 24-hour oral exposure, Dose is the drug dose; F % is the oral bioavailability.

Conclusion: The crystal forms of the compounds in the application have higher oral exposure, better oral bioavailability, and show good pharmacokinetic characteristics of an oral drug.

### Experimental Example 3: Study on DMPK in Cynomolgus Monkeys

### Experimental Purpose:

Male cynomolgus monkeys were used as test animals. After single administration, the plasma concentration of the compound was measured, and the pharmacokinetic behavior was evaluated.

### Experimental Operations:

Two healthy male cynomolgus monkeys were selected as a group. In the oral group, the vehicle was 20% HP-β-CD aqueous solution; after the compound to be tested was mixed with the vehicle, 4 mg/mL approximately clear solution was prepared by vortex and ultrasound. After oral administration at 20 mg/kg, the whole blood of cynomolgus monkey was collected for a certain period of time to prepare plasma; then the drug concentration was analyzed by LC-MS/MS method, and the pharmacokinetic parameters were calculated by Phoenix WinNonlin software (Pharsight, USA). The experimental results are shown in Table 27 below.

TABLE 27
PK detection results of the compound of the application

| | Cₘₐₓ | AUC₀₋ₗₐₛₜ | |
|---|---|---|---|
| compound No. | (nM) | (h*nmol/L) | F % |
| formula (O) compound | 1425 | 12765 | 22.1% |
| formula (II-1) compound | 2595 | 20676 | 35.5% |
| crystal Form A | | | |
| formula (II) compound | 2595 | 20676 | 37.7% |
| crystal Form E | | | |

Note:
HP-β-CD stands for hydroxypropyl-β-cyclodextrin; Cₘₐₓ is the maximum concentration; AUC₀₋ₗₐₛₜ is the 24-hour oral exposure; F % is the oral bioavailability.

Conclusion: The crystal forms of the compounds in the application have higher oral exposure, better oral bioavailability, the in vivo exposure of the crystal form is significantly higher than that of its free state, and the crystal forms show good pharmacokinetic characteristics of an oral drug.

1-73. (canceled)

74. A (i) compound of formula (O) or formula (I) and (ii) a pharmaceutically acceptable salt thereof, 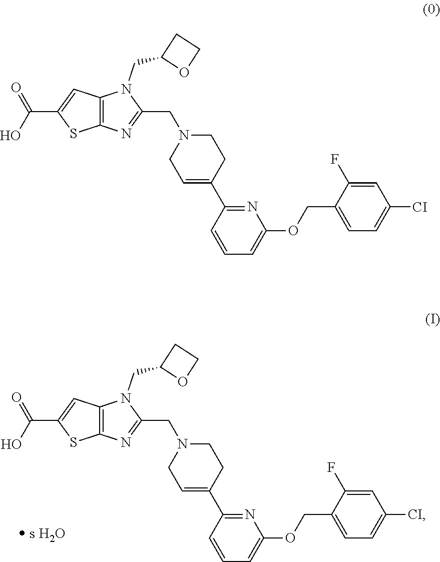
wherein s is selected from 0-3,
wherein the pharmaceutically acceptable salt is selected from the group consisting of: trometamol salt, phosphate, citrate, oxalate, maleate, L-tartrate, p-toluenesulfonate N, sodium salt, potassium salt, L-arginine salt, hydroxycholine salt, meglumine salt.

75. A method for preparing the pharmaceutically acceptable salt according to claim 74, comprising the step of carrying out a salt forming reaction of the formula (I) compound and an acid or base.

76. A pharmaceutical composition comprising the pharmaceutically acceptable salt according to claim 74 and optionally a pharmaceutically acceptable excipient.

77. A method of preventing and/or treating a metabolic disease, comprising:
administering the pharmaceutically acceptable salt according to claim 74 to a subject in need thereof.

78. A compound of Formula (II), 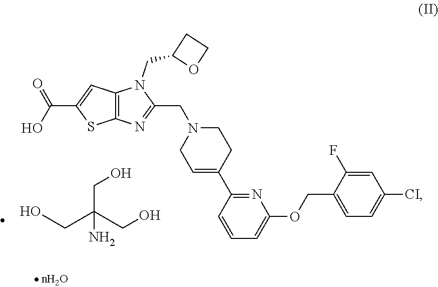
wherein n is selected from 0-12.

79. A crystal Form A of the compound according to claim 78, wherein the compound is of formula (II-1) 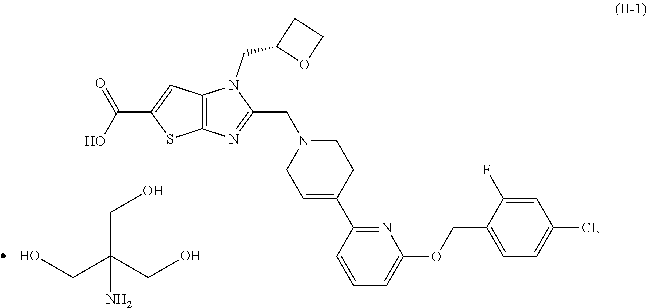
wherein the X-ray powder diffraction pattern of the crystal Form A has characteristic diffraction peaks at the following 2θ angles: 11.1426±0.2000°, 14.4804±0.2000°, and 21.4921±0.2000°.

80. A crystal Form E of the compound of formula (II) according to claim 78, wherein the X-ray powder diffraction pattern of the crystal Form E has characteristic diffraction peaks at the following 2θ angles: 18.8216±0.2000°, 20.0568±0.2000°, and 25.1936±0.2000°.

81. A compound of Formula (VII), 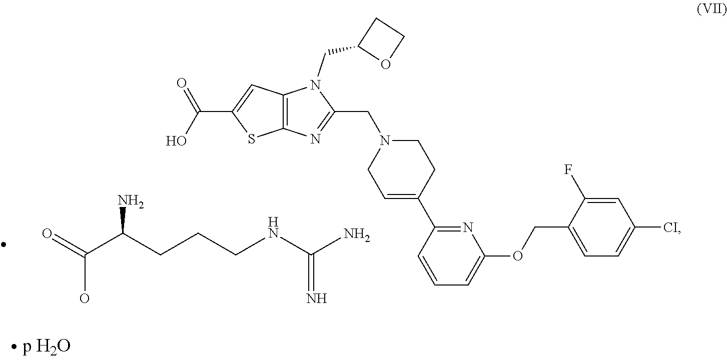
wherein p is selected from 0-3.

82. A crystal Form O of the compound of formula (VII) according to claim 81, wherein the X-ray powder diffraction pattern of the crystal Form O has characteristic diffraction peaks at the following 2θ angles: 9.485±0.200°, 11.273±0.200°, and 17.536±0.200°.

83. A crystal Form P of the compound of formula (VII) according to claim 81, wherein the X-ray powder diffraction pattern of the crystal Form P has characteristic diffraction peaks at the following 2θ angles: 12.481±0.200°, 14.965±0.200°, and 17.480±0.200°.

84. A compound of formula (VIII),

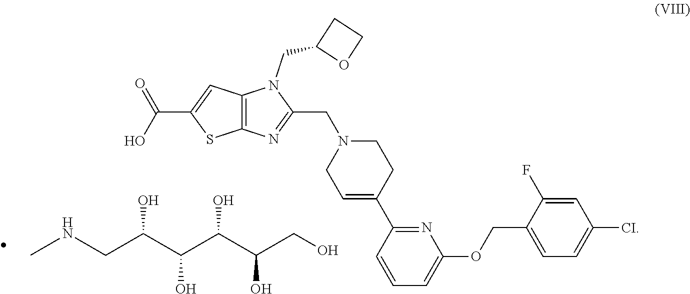

85. A crystal Form Q of the compound of formula (VIII) according to claim 84, wherein the X-ray powder diffraction pattern of the crystal Form Q has characteristic diffraction peaks at the following 2θ angles: 6.123±0.200°, 9.130±0.200°, and 12.123±0.200°.

86. A method of treating a metabolic disease, which comprises administering the compound according to claim 78 to a subject in need thereof.

87. A method of treating a metabolic disease, which comprises administering the compound according to claim 81 to a subject in need thereof.

88. A method of treating a metabolic disease, which comprises administering the compound according to claim 84 to a subject in need thereof.

89. A method of treating a metabolic disease, which comprises administering the crystal Form A according to claim 79 to a subject in need thereof.

90. A method of treating a metabolic disease, which comprises administering the crystal Form E according to claim 80 to a subject in need thereof.

91. A method of treating a metabolic disease, which comprises administering the crystal Form O according to claim 82 to a subject in need thereof.

92. A method of treating a metabolic disease, which comprises administering the crystal Form P according to claim 83 to a subject in need thereof.

93. A method of treating a metabolic disease, which comprises administering the crystal Form Q according to claim 85 to a subject in need thereof.
